SEQUENCE LISTING (25 sequences)

SEQ ID NO: 1 - RNA, 100 nt, synthetic construct
  source 1..100: mol_type = other RNA
atcacaactgtagctgcatttcaccaagaatgtagtttacagtcatgtactcaacatcaaccatatgtagttgatgacccgtgtcctattcacttctatt

SEQ ID NO: 2 - DNA, 45 bp, synthetic construct
  source 1..45: mol_type = other DNA
ctttaatacgactcactatagggtagaagtgaataggacacgggt

SEQ ID NO: 3 - DNA, 47 bp, synthetic construct
  source 1..47: mol_type = other DNA
ctttaatacgactcactatagggagaatgtagtttacagtcatgtac

SEQ ID NO: 4 - DNA, 45 bp, synthetic construct
  source 1..45: mol_type = other DNA
cttaattaaccctcactaaagggtagaagtgaataggacacgggt

SEQ ID NO: 5 - DNA, 23 bp, synthetic construct
  source 1..23: mol_type = other DNA
atcacaactgtagctgcatttca

SEQ ID NO: 6 - DNA, 47 bp, synthetic construct
  source 1..47: mol_type = other DNA
cttaattaaccctcactaaagggagaatgtagtttacagtcatgtac

SEQ ID NO: 7 - DNA, 55 bp, synthetic construct
  source 1..55: mol_type = other DNA
acgaatataccacataccaaaccttccttcgtcaactacatatggttgatgttga

SEQ ID NO: 8 - RNA, 100 nt, synthetic construct
  source 1..100: mol_type = other RNA
aagacggaccagagcgaaagcatttgccaagaatgttttcattaatcaagaacgaaagtcggaggttcgaagacgatcagataccgtcgtagttccgacc

SEQ ID NO: 9 - DNA, 43 bp, synthetic construct
  source 1..43: mol_type = other DNA
ctttaatacgactcactatagggggaactacgacggtatctga

SEQ ID NO: 10 - DNA, 17 bp, synthetic construct
  source 1..17: mol_type = other DNA
cggaccagagcgaaagc

SEQ ID NO: 11 - DNA, 48 bp, synthetic construct
  source 1..48: mol_type = other DNA
ctttaatacgactcactataggggccaagaatgttttcattaatcaag

SEQ ID NO: 12 - DNA, 50 bp, synthetic construct
  source 1..50: mol_type = other DNA
acgaatataccacataccaaaccttccttcgttcttcgaacctccgactt

SEQ ID NO: 13 - DNA, 43 bp, synthetic construct
  source 1..43: mol_type = other DNA
cttatttaggtgacactatagggggaactacgacggtatctga

SEQ ID NO: 14 - DNA, 43 bp, synthetic construct
  source 1..43: mol_type = other DNA
cttaattaaccctcactaaagggggaactacgacggtatctga

SEQ ID NO: 15 - DNA, 71 bp, synthetic construct
  source 1..71: mol_type = other DNA
ggcacgtacgaatataccacataccaaaccttccttcgtacgtgccgccaagaatgttttcattaatcaag

SEQ ID NO: 16 - RNA, 103 nt, synthetic construct
  source 1..103: mol_type = other RNA
gagaatccagcacacaagagtcaactggtgtggatgggatgccattctgccgcatttgaagatctaagagtattgagcttcatcaaagggacgaaggtggtcc

SEQ ID NO: 17 - DNA, 40 bp, synthetic construct
  source 1..40: mol_type = other DNA
cttaattaaccctcactaaagggggaccaccttcgtccct

SEQ ID NO: 18 - DNA, 40 bp, synthetic construct
  source 1..40: mol_type = other DNA
taccccctgcatacactaatgagaatccagcacacaagag

SEQ ID NO: 19 - DNA, 40 bp, synthetic construct
  source 1..40: mol_type = other DNA
cttaattaaccctcactaaagggctggtgtggatgggatg

SEQ ID NO: 20 - DNA, 56 bp, synthetic construct
  source 1..56: mol_type = other DNA
acgaatataccacataccaaaccttccttcgtgctcaatactcttagatcttcaaa

SEQ ID NO: 21 - DNA, 97 bp, synthetic construct
  source 1..97: mol_type = other DNA
atccgcctgagtagtatgctcgcaagagtgaaacttaaaggaattgacgggaacccgcacaagcggtggagcatgtggtttaatttgaagatacgcg

SEQ ID NO: 22 - DNA, 46 bp, synthetic construct
  source 1..46: mol_type = other DNA
cttaattaaccctcactaaagggcgcgtatcttcaaattaaaccac

SEQ ID NO: 23 - DNA, 19 bp, synthetic construct
  source 1..19: mol_type = other DNA
atccgcctgagtagtatgc

SEQ ID NO: 24 - DNA, 48 bp, synthetic construct
  source 1..48: mol_type = other DNA
cttaattaaccctcactaaagggcaagagtgaaacttaaaggaattga

SEQ ID NO: 25 - DNA, 48 bp, synthetic construct
  source 1..48: mol_type = other DNA
acgaatataccacataccaaaccttccttcgtcaccgcttgtgcgggt

(19) United States
(12) Patent Application Publication
(10) Pub. No.: US 2025/0354205 A1
(43) Pub. Date: Nov. 20, 2025

(54) NUCLEIC ACID DETECTION

(71) Applicant: IMPERIAL COLLEGE INNOVATIONS LIMITED, London (GB)

(72) Inventors: David Santiago Rueda, London (GB); Haruki lino, London (GB)

(73) Assignee: IMPERIAL COLLEGE INNOVATIONS LIMITED, London (GB)

(21) Appl. No.: 18/836,861

(22) PCT Filed: Feb. 8, 2023
(86) PCT No.: PCT/GB2023/050275

§ 371 Date: Aug. 8, 2024

(30) Foreign Application Priority Data
Feb. 9, 2022 (GB) 2201656.2

(51) Int. Cl. C12Q 1/6844; C12Q 1/689; C12Q 1/70

(52) U.S. Cl. CPC C12Q 1/6844; C12Q 1/689; C12Q 1/701; C12Q 2600/158

(57) ABSTRACT

The invention relates to nucleic acid detection, and particularly, although not exclusively, to rapid, one-pot, single-step methods and associated kits for amplifying and detecting target nucleic acid sequences in a sample obtained from a subject. The methods and kits can be used to detect pathogenic nucleic acid sequences in a sample, such as a blood or saliva sample, via the selective amplification of the target nucleic acids, to provide a point of care diagnosis for infectious diseases, or other RNA-related diseases, such as cancer.

### CROSS-REFERENCE TO RELATED APPLICATIONS

This application is a National Phase Entry of International Application No. PCT/GB2023/050275, filed Feb. 8, 2023, which designated the U.S. and which includes a claim of priority to United Kingdom patent application No. 2201656.2, filed Feb. 9, 2022, the entirety of each of which is hereby incorporated by reference.

The present invention relates to nucleic acid detection, and particularly, although not exclusively, to rapid, one-pot, single-step methods and associated kits for amplifying and detecting target nucleic acid sequences in a sample obtained from a subject. The methods and kits can be used to detect pathogenic nucleic acid sequences in a sample, such as a blood or saliva sample, via the selective amplification of the target nucleic acids, to provide a point of care diagnosis for infectious diseases, or other RNA-related diseases, such as cancer.

During the COVID-19 pandemic, it has become clear that detecting and tracing infection cases is critical for controlling the spread of viral transmission. For this reason, point of care diagnosis plays a pivotal role, and diagnostic methods should meet the criteria of rapidity, simplicity, sensitivity, affordability, and portability.

Despite the huge effort for establishing a rapid and portable test, reverse transcription polymerase chain reaction (RT-PCR) tests are still the gold standard method that are adopted by WHO, and give the best limit of detection (LOD) (˜0.1 copies/μL) among available techniques. However, the method is not suitable in terms of a rapid point-of-care test, because the collected patient samples must be transported to equipped laboratories, and analysed with specialized techniques and equipment. Accordingly, RT-PCR tests require several hours or days to be completed, and do not meet the criteria of rapidity. Additionally, the high cost of thermocyclers potentially further limits the diagnosis.

To solve these limitations of RT-PCR tests, several alternative methods have been established. Reverse transcription loop mediated isothermal amplification (RT-LAMP) is an isothermal reaction method, and the target amplification is performed at DNA melting temperature (60-65° C.) with a specialised DNA polymerase (Bst DNA polymerase) and several sets of primers. Since the reaction is so rapid and produces pyrophosphates, the pH change can be used as a readout. DNA staining can help increase the LOD to ˜10 copies/μL. Although the sensitivity gets worse, it is possible that this method can skip the RNA isolation step and can directly detect viral RNAs in the heated positive specimens. Currently, RT-LAMP has been accepted as an alternative of RT-PCR and is indeed used as a point of care diagnosis.

Antigen-based tests performed by lateral flow strips are commonly used as quick tests, taking approximately 15 minutes. Instead of amplifying the pathogen signals with enzymatic reactions, the antibodies on the strip simply detect the antigens of the pathogens. As a result, the LOD is low (>100 copies/μL), and normally only active infection can be detected.

Nucleic acid sequence-based amplification (NASBA) is a molecular biology technique that produces multiple transcribed copies of RNA of a specific DNA or RNA fragment, using three enzymes and two primers. First, when amplifying a target RNA sequence, a primer containing a T7 promoter hybridizes to the target RNA and is extended using dNTPs by a reverse transcriptase (RT), forming a cDNA copy of the template RNA. RNase H then destroys the hybridized RNA to leave the bare cDNA. A second primer then hybridizes to the cDNA and the RT synthesises another DNA strand from the attached primer, producing a double stranded DNA. T7 RNA polymerase binds to the promoter region on the double strand and then transcribes RNA using NTPs. As multiple copies of RNA are made, free primer can continue to hybridize, be extended, and produce more template. This results in exponential amplification of the DNA template and RNA product. Alternatively, when amplifying a target DNA sequence, a first primer hybridises to the DNA sequence (following a first denaturing step) and is extended by RT. A second primer is added and following a second denaturation step, the primer hybridises to the extension product of the first primer and is extended, producing a double stranded DNA sequence. T7 RNA polymerase then binds to the promoter and transcribes RNA.

Mango is a fluorescent RNA aptamer that can be used to label RNAs of interest. Mango fluoresces upon binding to a thiazole orange-based ligand (TO1-Biotin), providing a fluorescent readout of the transcription of a specific DNA sequence and allowing the technique to be used to identify pathogens in biological samples such as saliva or blood.

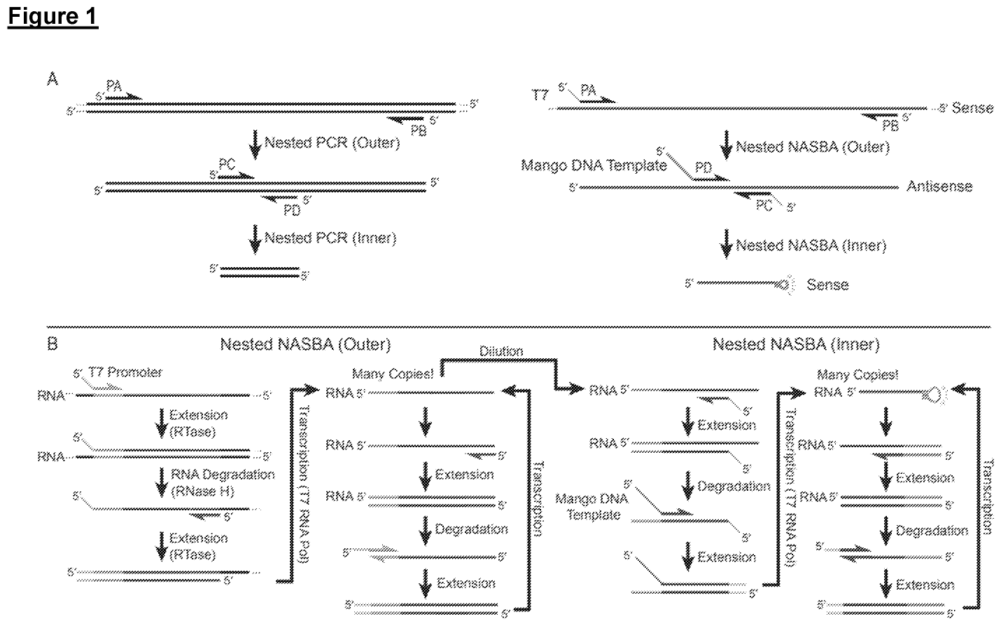

The NASBA molecular biology technique has been combined with fluorogenic aptamers, such as Mango, to produce the nested Mango-NASBA assay for amplifying and detecting nucleic acid sequences (see FIG. 1). This nested Mango-NASBA assay amplifies RNA sequences that are recognised by two sets of primers (outer primers: PA/PB or P1/P2, and inner primers: PC/PD or P3/P4). However, the nested Mango-NASBA assay requires that a saliva sample is first pre-treated to extract the pathogenic nucleic acids, before two sequential nested amplification reactions are performed. Accordingly, the current nested Mango-NASBA assay for detecting nucleic acids does not meet the criteria of rapidity, simplicity and portability.

There is, therefore, a need to provide an improved method of rapidly detecting nucleic acids, to provide a point of care diagnosis for infectious diseases.

The inventors have modified nucleic acid detection methods, and surprisingly demonstrated that, firstly, by carefully controlling the amounts of reagents and primer, the two nested amplifications steps of the Mango-NASBA method can be performed in parallel in a single tube. Secondly, by changing the RNA polymerase used in the original assay, the detection of the Mango fluorescent signal can be improved and stabilised.

Therefore, according to a first aspect of the invention, there is provided a method of detecting a target nucleic acid sequence in a sample obtained from a subject, the method comprising:
amplifying the target nucleic acid sequence by nucleic acid sequence based amplification (NASBA), wherein the amplification comprises contacting the sample with:
   (a) one or more primers, wherein at least one of the primers comprises a fluorescent molecule;
   (b) one or more polymerases; and
   (c) a plurality of deoxynucleotide triphosphates (dNTPs) and/or nucleotide triphosphates (NTPs), and
detecting the target nucleic acid sequence, wherein a fluorescent signal indicates the presence of the target nucleic acid sequence in the sample, wherein the amplification step comprises at least one of the following conditions:
   (i) contacting the sample with between 1 and 59 mM Tris HCl;
   (ii) contacting the sample with spermidine;
   (iii) a pH of between 6.8 and 9.0;
   (iv) a fixed temperature; and/or
   (v) the polymerase is T3 RNA polymerase.

According to a second aspect of the invention, there is provided a method of diagnosing or prognosing an infectious disease in a subject, the method comprising detecting a target nucleic acid sequence in a sample obtained from a subject, comprising:
amplifying the target nucleic acid sequence by nucleic acid sequence based amplification (NASBA), wherein the amplification comprises contacting the sample with:
   (a) one or more primers, wherein at least one of the primers comprises a fluorescent molecule;
   (b) one or more polymerases; and
   (c) a plurality of deoxynucleotide triphosphates (dNTPs) and/or nucleotide triphosphates (NTPs), and
detecting the target nucleic acid sequence, wherein a fluorescent signal indicates that the subject suffers from an infectious disease or has a negative prognosis, wherein the amplification step comprises at least one of the following conditions:
   (i) contacting the sample with between 1 and 59 mM Tris HCl;
   (ii) contacting the sample with spermidine;
   (iii) a pH of between 6.8 and 9.0;
   (iv) a fixed temperature; and/or
   (v) the polymerase is T3 RNA polymerase.

According to a third aspect of the invention, there is provided a kit for detecting a target nucleic acid sequence in a sample obtained from a subject, the kit comprising:
reagents for amplifying the target nucleic acid sequence by nucleic acid sequence based amplification (NASBA), wherein the reagents comprise:
   (a) one or more primers, wherein at least one of the primers comprises a fluorescent molecule;
   (b) one or more polymerases; and
   (c) a plurality of deoxynucleotide triphosphates (dNTPs) and/or nucleotide triphosphates (NTPs), and
means for detecting the target nucleic acid sequence, wherein a fluorescent signal indicates the presence of the target nucleic acid sequence in the sample, wherein the kit comprises at least one of the following:
   (i) between 1 and 59 mM Tris HCl;
   (ii) spermidine;
   (iii) a pH of between 6.8 and 9.0;
   (iv) is used at a fixed temperature; and/or
   (v) the polymerase is T3 RNA polymerase.

As discussed above, the inventors were surprised to observe that by carefully controlling the amounts of reagents and primer, the two nested amplifications steps of the prior art NASBA method can be performed in parallel in a single tube. Also, by changing the RNA polymerase used in the original assay, the detection of the fluorescent molecule's signal can be improved and stabilised. Advantageously, the result of these improvements is that the reaction becomes single-step, single-vessel and single-temperature, taking less than 30 minutes. The reaction can also be performed on saliva stored in conventional viral transport buffer, without any pre-treatment or RNA extraction. As such, the improved NASBA method and kit of the claimed invention is a rapid, portable and highly adaptable technique, well-suited for infection control in a variety of settings. Additionally, by using multiple fluorescent molecules targeting multiple sequences, the technique can be easily adapted to test for multiple pathogens in a single reaction.

In one embodiment, the method and/or kit comprises at least conditions: (i) and (ii); (i) and (iii); (i) and (iv); (i) and (v); (ii) and (iii); (ii) and (iv); (ii) and (v); (iii) and (iv); (iii) and (v); or (iv) and (v).

Alternatively, in another embodiment, the method and/or kit comprises at least conditions: (i), (ii) and (iii); (i), (ii) and (iv); (i), (ii) and (v); (i), (iii) and (iv); (i), (iii) and (v); (i), (iv) and (v); (ii), (iii) and (iv); (ii), (iv) and (v); (ii), (iv) and (v); or (iii), (iv) and (v).

Alternatively, in another embodiment, the method and/or kit comprises at least conditions: (i), (ii), (iii) and (iv); (i), (ii), (iii) and (v); (i), (ii), (iv) and (v); (i), (iii), (iv) and (v); or (ii), (iii), (iv) and (v).

Alternatively, in another embodiment, the method and/or kit comprises all of conditions (i), (ii), (iii), (iv) and (v).

Advantageously, with the method according to the first and second aspect, or the kit according to the third aspect, there is no need to extract RNA from the sample prior to performing the amplification reaction. Accordingly, in a preferred embodiment, the method and/or kit does not comprise extracting or purifying the nucleic acid from the sample, or means for extracting or purifying the nucleic acid from the sample.

Preferably, the method is conducted in a single step and/or a single pot or container. This is a significant advance of prior art methods which require multiple steps carried out in multiple containers.

The term “primer” designates, within the context of the present invention, a nucleotide sequence that can hybridize specifically to the target nucleic acid sequence and serve to initiate amplification.

Primers of the invention may be a single-stranded nucleotide sequence, with a length of between 10 and 200 nucleotides, between 10 and 180, between 10 and 160, between 10 and 140, between 10 and 120, between 10 and 110, between 10 and 100, between 10 and 80 nucleotides, between 10 and 70 nucleotides, between 10 and 60 nucleotides, between 10 and 50 nucleotides, between 10 and 40 nucleotides, between 10 and 30 nucleotides, or between 10 and 20 nucleotides.

Preferably, the primers of the invention have a length of between 15 and 60 nucleotides, between 25 and 60 nucleotides, between 35 and 60 nucleotides, between 45 and 60 nucleotides, between 55 and 60 nucleotides, between 15 and 50 nucleotides, between 25 and 50 nucleotides, between 35 and 50 nucleotides, between 45 and 50 nucleotides, between 15 and 40 nucleotides, between 25 and 40 nucleotides, between 35 and 40 nucleotides, between 15 and 30 nucleotides, or between 25 and 30 nucleotides.

Preferably, the primers of the invention have a length of between 10 and 30 nucleotides, between 12 and 28 nucleotides, between 14 and 26 nucleotides, between 16 and 24 nucleotides, or between 18 and 22 nucleotides. In another preferred embodiment, the primers of the invention have a length of between 35 and 55 nucleotides, between 37 and 53 nucleotides, between 39 and 51 nucleotides, between 41 and 49 nucleotides, or between 43 and 47 nucleotides.

In one embodiment, the one or more primers comprises a first and second primer (a forward primer and a reverse primer). Preferably, in this embodiment, the first and second primer form a first primer pair. The forward primer is designed so that it is complementary to a sequence of nucleotides upstream of the target sequence, whilst the reverse primer is designed so that it is complementary to a sequence of nucleotides downstream of the target sequence.

Preferably, the first and second primer are capable of hybridising to at least a portion of the target nucleic acid sequence, or reverse-complement thereof, and performing a first amplification reaction to produce a first amplification product.

Alternatively, the amplification reaction may be “nested”. This nested reaction involves dilution of the first amplification product, prior to performing a subsequent amplification, improving the sensitivity and specificity and reducing the amplification artefacts.

Accordingly, in another embodiment, the one or more primers comprises a first, second, third and fourth primer. Preferably, in this embodiment, the first and second primer (forward and reverse primer) form a first primer pair, and the second and third primer (forward and reverse primer) form a second primer pair.

Preferably, the first and second primer are capable of hybridising to at least a portion of the target nucleic acid sequence, or reverse-complement thereof, and performing a first amplification reaction to produce a first amplification product. Preferably, the second and third primer are capable of hybridising to at least a portion of the first amplification product, or reverse-complement thereof, and performing a second amplification reaction to produce a second amplification product.

As discussed in Example 3, the inventors discovered that the nested Mango-NASBA reaction only worked when the same RNA promoter sequence was used in the outer (P1) and inner (P3) primer. Accordingly, in a preferred embodiment, the first and third primer comprise a polymerase promoter comprising an identical nucleotide sequence.

In one embodiment, the fluorescent molecule may be a molecular beacon probe. Molecular beacon probes are single-stranded oligonucleotides having a stem-and-loop structure. A fluorophore and a quencher are covalently linked to either end of the molecular beacon, with the stem keeping them in close proximity, causing the fluorescence of the fluorophore to be quenched. When the molecular beacon hybridizes to its target it undergoes a conformational change that separates the fluorophore and the quencher, allowing the fluorophore to emit fluorescent light. The fluorophore may be any fluorophore well known to the skilled person, for example, fluorescein (FAM), 6-carboxy-X-rhodamine (ROX), and Cascade Blue (CB).

Preferably, the fluorescent molecule is a fluorogenic aptamer. A fluorogenic aptamer is a nucleic acid molecule that can bind to a ligand (a fluorophore) with high selectivity and specificity, and generate a fluorescent signal.

In one embodiment, the fluorogenic aptamer may be selected from a group of aptamers consisting of: Mango, Pepper, Peach, Broccoli, Corn, Spinach, Spinach2, Carrot, Radish, RT aptamer, hemin-binding G-quadruplex DNA and RNA aptamers, and a malachite green binding aptamer. Examples of fluorogenic aptamers are discussed in Ouellet, Jonathan. “RNA fluorescence with light-up aptamers.” Frontiers in chemistry 4 (2016): 29, Dolgosheina, Elena V., et al. “RNA mango aptamer-fluorophore: a bright, high-affinity complex for RNA labeling and tracking.” ACS chemical biology 9.10 (2014): 2412-2420, Strack, Rita L., Matthew D. Disney, and Samie R. Jaffrey. “A superfolding Spinach2 reveals the dynamic nature of trinucleotide repeat-containing RNA.” Nature methods 10.12 (2013): 1219-1224, Filonov, Grigory S., et al. “Broccoli: rapid selection of an RNA mimic of green fluorescent protein by fluorescence-based selection and directed evolution.” Journal of the American Chemical Society 136.46 (2014): 16299-16308, and Song, Wenjiao, et al. “Imaging RNA polymerase III transcription using a photostable RNA-fluorophore complex.” Nature chemical biology 13.11 (2017): 1187-1194.

Most preferably, the fluorogenic aptamer is a Mango aptamer, such as a Mango RNA aptamer. The Mango RNA aptamer binds to a thiazole orange-based ligand (T01-Biotin), generating a fluorescent signal. Accordingly, in one embodiment, the method comprises contacting the sample with thiazole orange-based ligand (T01-Biotin), and/or the kit comprises thiazole orange-based ligand (T01-Biotin). Preferably, the Mango RNA aptamer is a Mango II, Mango III or Mango IV RNA aptamer.

The first, second, third or fourth primer may comprise the fluorescent molecule. Preferably, the fluorescent molecule is positioned at the 3′ end of the first, second, third or fourth primer. Most preferably, when the amplification reaction is nested, the third or fourth primer comprises the fluorescent molecule.

As discussed in Example 1, the inventors determined the optimal length of Mango primer stem nucleotides, and discovered that the 5-mer stem primer set indicated the best amplification ratio between plus and minus template RNAs.

Accordingly, in one embodiment, the primer comprising the fluorescent molecule comprises a terminal stem structure of between 2 bp and 15 bp, between 3 bp and 14 bp, between 4 bp and 13 bp, between 5 and 12 bp, between 6 and 11 bp, or between 7 and 10 bp.

In one embodiment, the primer comprising the fluorescent molecule comprises a terminal stem structure of between 2 bp and 13 bp, between 2 bp and 11 bp, between 2 bp and 9 bp, between 2 bp and 7 bp, between 2 bp and 5 bp, between 3 bp and 15 bp, between 3 bp and 13 bp, between 3 bp and 11 bp, between 3 bp and 9 bp, between 3 bp and 7 bp, between 3 bp and 5 bp, between 4 bp and 15 bp, between 4 bp and 13 bp, between 4 bp and 11 bp, between 4 bp and 9 bp, or between 4 bp and 7 bp.

Preferably, the primer comprising the fluorescent molecule comprises a terminal stem structure of between 2 bp and 8 bp, or between 3 bp and 7 bp, or between 4 bp and 6 bp. Most preferably, the primer comprising the fluorescent molecule comprises a terminal stem structure of 5 bp.

Additionally, as discussed in Example 4, the inventors conducted systematic titration of primers to determine the optimal primer concentration. The inventors discovered that the Mango signals were efficiently amplified when the concentration of the outer primers (i.e. the first primer pair) was lower than that of the inner primers (i.e. the second primer pair).

Accordingly, in a preferred embodiment, the concentration of the first and second primer (i.e. the first primer pair) is lower than the concentration of the third and fourth primer (i.e. the second primer pair).

For example, in one embodiment, the concentration of the first and second primer may be between 1 and 50 nM, between 5 and 45 nM, between 10 and 40 nM, between 15 and 35 nM, or between 20 and 30 nM.

In one embodiment, the concentration of the first and second primer may be between 1 and 50 nM, between 1 and 45 nM, between 1 and 40 nM, between 1 and 35 nM, between 1 and 30 nM, between 1 and 25 nM, between 1 and 20 nM, between 1 and 15 nM, between 1 and 10 nM, or between 1 and 5 nM.

In another embodiment, the concentration of the first and second primer is between 5 and 50 nM, between 10 and 50 nM, between 15 and 50 nM, between 20 and 50 nM, between 25 and 50 nM, between 30 and 50 nM, between 35 and 50 nM, between 40 and 50 nM, or between 45 and 50 nM. Preferably, the concentration of the first and second primer is between 22 and 28 nM, or between 24 and 26 nM. Most preferably, the concentration of the first and second primer is 25 nM.

The concentration of the third and fourth primer may be between 10 and 150 nM, between 20 and 140 nM, between 30 and 130 nM, between 40 and 120 nM, between 50 and 110 nM, between 60 and 100 nM, or between 70 and 90 nM.

In one embodiment, the concentration of the third and fourth primer may be between 20 and 150 nM, 30 and 150 nM, 40 and 150 nM, 50 and 150 nM, 60 and 150 nM, 70 and 150 nM, 80 and 150 nM, 90 and 150 nM, 100 and 150 nM, 110 and 150 nM, 120 and 150 nM, 130 and 150 nM, or 140 and 150 nM. In another embodiment, the concentration of the third and fourth primer may be between 10 and 140 nM, 10 and 130 nM, 10 and 120 nM, 10 and 110 nM, 10 and 100 nM, 10 and 90 nM, 10 and 80 nM, 10 and 70 nM, 10 and 60 nM, 10 and 50 nM, 10 and 40 nM, 10 and 30 nM, or 10 and 20 nM. Most preferably, the concentration of the third and fourth primer is 100 nM.

The inventors also discovered that excess levels of primers, e.g. over 100 nM, resulted in the reduction of Mango signal amplifications. Accordingly, in a preferred embodiment, the concentration of the one or more primers is 100 nM or less.

Furthermore, as discussed in Example 14, the inventors discovered that optimising the melting temperature (Tm) of the primers, improves the amplification of Mango signals, and the signal difference in the presence and absence of template RNAs was maximised when all primer melting temperatures were 51° C.

Accordingly, in one embodiment, the melting temperature of the one or more primers is between 48 and 54° C., between 49 and 53° C., or between 50 and 52° C. Most preferably, the melting temperature of the one or more primers is 51° C.

For example, in an embodiment where the one or more primers comprises a first and second primer, preferably the melting temperature of the first and second primer is 51° C. In an embodiment where the one or more primers comprises a third and fourth primer, preferably the melting temperature of the third and fourth primer is 51° C. Alternatively, in an embodiment where the one or more primers comprises a first, second, third and fourth primer, preferably the melting temperature of the first, second, third and fourth primer is 51° C.

As discussed above, in some embodiments, the method comprises one single amplification reaction. In this embodiment, the amplification comprises contacting the sample with one polymerase.

Alternatively, in embodiments in which the amplification reaction is nested (i.e. comprises two amplification reactions), the amplification comprises contacting the sample with a first and second polymerase.

Preferably, the one or more polymerases is an RNA polymerase. Even more preferably, the one or more polymerases is a T3 RNA polymerase. Alternatively, in an embodiment in which the one or more polymerases is not a T3 RNA polymerase, the one or more polymerase may be selected from a group of polymerases consisting of: T7 RNA polymerase, and SP6 RNA polymerase.

Therefore, in an embodiment in which the method comprises one single amplification reaction, the amplification comprises contacting the sample with T3 RNA polymerase. As discussed in Example 3, the inventors discovered that the nested Mango-NASBA reaction only worked when the same RNA polymerase was used in the first and second amplification reaction.

Accordingly, in embodiments in which the amplification comprises contacting the sample with a first and second polymerase, preferably the first and second polymerase are the same type of polymerase. Most preferably, the first and second polymerase are a T3 RNA polymerase.

The target nucleic acid sequence may be a DNA or RNA sequence. In a preferred embodiment, the target nucleic acid sequence is an RNA sequence. The target nucleic acid sequence may be single-stranded or double-stranded, or a combination of single- and double-stranded. For example, the target nucleic acid sequence may be selected from chromosomal DNA, mitochondrial DNA, messenger RNA, transfer RNA, ribosomal RNA, small nuclear RNA, micro RNA, small-interfering RNA, viral RNA and extrachromosomal DNA.

In some embodiments, the target nucleic acid sequence may be from a virus, a bacteria, a mycoplasma, a fungus, an animal, a plant, an alga, a parasite, or a protozoan. Preferably, the target nucleic acid sequence is from a virus or a bacterium.

In one embodiment, the target nucleic acid sequence may be from an RNA-related disease, such as cancer.

The target nucleic acid sequence may be between 10 and 1000 nucleotides, between 20 and 900 nucleotides, between 30 and 800 nucleotides, between 40 and 700 nucleotides, between 50 and 600 nucleotides, between 60 and 500 nucleotides, between 70 and 400 nucleotides, between 80 and 300 nucleotides, or between 90 and 200 nucleotides in length.

In one embodiment, the target nucleic acid sequence may be between 10 and 800 nucleotides, between 10 and 600 nucleotides, between 10 and 400 nucleotides, between 10 and 200 nucleotides, between 20 and 1000 nucleotides, between 20 and 800 nucleotides, between 20 and 600 nucleotides, between 20 and 400 nucleotides, between 20 and 200 nucleotides, between 30 and 1000 nucleotides, between 30 and 800 nucleotides, between 30 and 600 nucleotides, between 30 and 400 nucleotides, between 30 and 200 nucleotides, between 40 and 1000 nucleotides, between 40 and 800 nucleotides, between 40 and 600 nucleotides, between 40 and 400 nucleotides, between 40 and 200 nucleotides, between 50 and 1000 nucleotides, between 50 and 800 nucleotides, between 50 and 600 nucleotides, between 50 and 400 nucleotides, between 50 and 200 nucleotides, between 60 and 1000 nucleotides, between 60 and 800 nucleotides, between 60 and 600 nucleotides, between 60 and 400 nucleotides, between 60 and 200 nucleotides, between 70 and 1000 nucleotides, between 70 and 800 nucleotides, between 70 and 600 nucleotides, between 70 and 400 nucleotides, between 70 and 200 nucleotides, between 80 and 1000 nucleotides, between 80 and 800 nucleotides, between 80 and 600 nucleotides, between 80 and 400 nucleotides, between 80 and 200 nucleotides, between 90 and 1000 nucleotides, between 90 and 800 nucleotides, between 90 and 600 nucleotides, between 90 and 400 nucleotides, between 90 and 200 nucleotides, between 100 and 1000 nucleotides, between 100 and 800 nucleotides, between 100 and 600 nucleotides, between 100 and 400 nucleotides, or between 100 and 200 nucleotides in length.

Preferably, the target nucleic acid sequence may be between 40 and 160 nucleotides, between 50 and 150 nucleotides, between 60 and 140 nucleotides, between 70 and 130 nucleotides, between 80 and 120 nucleotides, or between 90 and 110 nucleotides in length.

The virus may be a DNA virus or an RNA virus. The virus may be selected from one of the following: African horse sickness virus; African swine fever virus; Akabane virus; Bhanja virus; Caliciviruses (e.g., human enteric viruses such as norovirus and sapovirus), Cercopithecine herpesvirus 1; Chikungunya virus; Classical swine fever virus; coronaviruses (e.g., Severe Acute Respiratory Syndrome (SARS), Middle East Respiratory Syndrome (MERS), Severe Acute Respiratory Syndrome coronavirus 2 (SARS-CoV-2)); Dengue viruses such as serotypes 1 (DENV1) and 3 (DENV3), and related viruses such as the chikungunya virus (CHIKV); Dugbe virus; Ebola viruses; Encephalitic viruses such as Eastern equine encephalitis virus, Japanese encephalitis virus, Murray Valley encephalitis, and Venezuelan equine encephalitis virus; Equine morbillivirus; flaviruses, Flexal virus; Foot and mouth disease virus; Germiston virus; Goat pox virus; Hantaan or other Hanta viruses; Hendra virus; human immunodeficiency virus (HIV); influenza viruses (e.g., H1 N1, H5N1, Avian influenza virus); Lassa fever virus; Louping ill virus; Lymphocytic choriomeningitis virus; Poliovirus; Potato virus; pox viruses; South American hemorrhagic fever viruses; Variola major virus (Smallpox virus); Vesicular stomatitis virus; West Nile virus; Yellow fever virus; human-pathogenic flaviviruses such Zika virus.

The bacterium may selected from one of the following: Aeromonas hydrophila; Bacillus anthracis; Bacillus cereus; botulinum neurotoxin producing species of Clostridium; Brucella abortus; Brucella melitensis; Brucella suis; Burkholderia mallei (formally Pseudomonas mallei); Burkholderia pseudomallei (formerly Pseudomonas pseudomallei); Campylobacter jejuni; Chlamydia psittaci; Clostridium botulinum; Clostridium botulinum; Clostridium perfringens; Coccidioides immitis; Coccidioides posadasii; Cowdria ruminantium; Coxiella burnetii; Enterovirulent Escherichia coli group (EEC Group) such as Escherichia coli-enterotoxigenic (ETEC), Escherichia coli-enteropathogenic (EPEC), Escherichia coli-0157.′H7 enterohemorrhagic (EHEC), and Escherichia coli-enteroinvasive (EIEC); Ehrlichia spp. such as Ehrlichia chaffeensis; Francisella tularensis; Legionella pneumophilia; Liberobacter africanus; Liberobacter asiaticus; Listeria monocytogenes; miscellaneous enterics such as Klebsiella, Enterobacter, Proteus, Citrobacter, Aerobacter, Providencia, and Serratia; Mycobacterium bovis; Mycobacterium tuberculosis; Mycoplasma capricolum; Mycoplasma mycoides ssp mycoides; Rickettsia prowazekii; Rickettsia rickettsii; Salmonella spp.; Schlerophthora rayssiae varzeae; Shigella spp.; Staphylococcus aureus; Streptococcus; Synchytrium endobioticum; Vibrio cholerae non-01; Vibrio cholerae 01; Vibrio parahaemolyticus and other Vibrios; Vibrio vulnificus; Xanthomonas oryzae; Xylella fastidiosa; Yersinia enterocolitica and Yersinia pseudotuberculosis; or Yersinia pestis.

In one embodiment, the target nucleic acid sequence is from SARS-CoV-2. One embodiment of the SARS-CoV-2 target nucleic acid sequence is referred to herein as SEQ ID No: 1, as follows:

[SEQ ID No: 1]
AUCACAACUG UAGCUGCAUU UCACCAAGAA UGUAGUUUAC
AGUCAUGUAC UCAACAUCAA CCAUAUGUAG UUGAUGACCC
GUGUCCUAUU CACUUCUAUU

Thus, in a preferred embodiment, the SARS-CoV-2 target nucleic acid sequence comprises or consists of a nucleotide sequence substantially set out as SEQ ID No: 1, or a fragment or variant thereof.

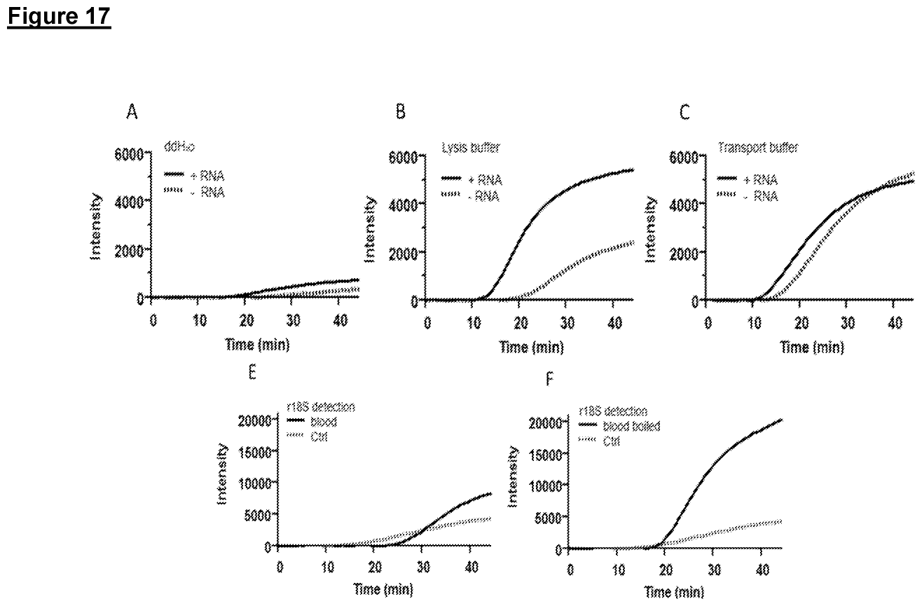

Preferably, in this embodiment, the one or more primers are capable of hybridising to the nucleotide sequence as substantially set out in SEQ ID No: 1, or a fragment or variant thereof. Examples of such primers are listed in FIG. 18.

Accordingly, in one embodiment, the nucleotide sequences encoding the one or more SARS-CoV-2 primers are referred to herein as SEQ ID Nos: 2 to 7, as follows:

[SEQ ID No: 2]
CTTTAATACG ACTCACTATA GGGTAGAAGT GAATAGGACA CGGGT
[SEQ ID No: 3]
CTTTAATACG ACTCACTATA GGGAGAATGT AGTTTACAGT
CATGTAC
[SEQ ID No: 4]
CTT AAT TAA CCC T CA C TA AAG GGT AGA AGT GAA T
AGG ACA CGG GT
[SEQ ID No: 5]
ATCACAACTG TAGCTGCATT TCA
[SEQ ID No: 6]
CTTAATTAA CCCTCACTAA AGGGAGAATG TAGTTTACAG
TCATGTAC
[SEQ ID No: 7]
ACGAATATAC CACATACCAA ACCTTCCTTC GTCAACTACA
TATGGTTGAT GTTGA

Thus, in a preferred embodiment, the one or more primers are encoded by a nucleotide sequence substantially set out in any one of SEQ ID Nos: 2 to 7, or a fragment or variant thereof.

In another embodiment, the target nucleic acid sequence is from 18S-rRNA. One embodiment of the 18S-rRNA target nucleic acid sequence is referred to herein as SEQ ID No: 8, as follows:

[SEQ ID No: 8]
AAGACGGACC AGAGCGAAAG CAUUUGCCAA GAAUGUUUUC
AUUAAUCAAG AACGAAAGUC GGAGGUUCGA AGACGAUCAG
AUACCGUCGU AGUUCCGACC

Thus, in a preferred embodiment, the 18S-rRNA target nucleic acid sequence comprises or consists of a nucleotide sequence substantially set out as SEQ ID No: 8, or a fragment or variant thereof.

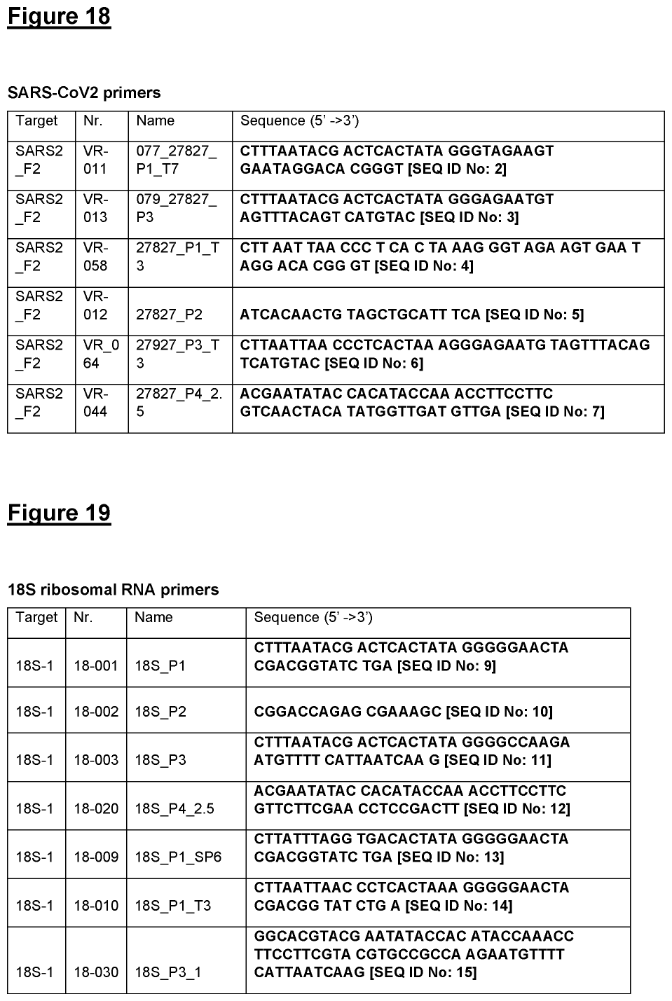

Preferably, in this embodiment, the one or more primers are capable of hybridising to the nucleotide sequence as substantially set out in SEQ ID No: 8, or a fragment or variant thereof. Examples of such primers are listed in FIG. 19.

Accordingly, in one embodiment, the nucleotide sequences encoding the one or more 18S-rRNA primers are referred to herein as SEQ ID Nos: 9 to 15, as follows:

[SEQ ID No: 9]
CTTTAATACG ACTCACTATA GGGGGAACTA CGACGGTATC TGA
[SEQ ID No: 10]
CGGACCAGAG CGAAAGC
[SEQ ID No: 11]
CTTTAATACG ACTCACTATA GGGGCCAAGA ATGTTTT
CATTAATCAA G
[SEQ ID No: 12]
ACGAATATAC CACATACCAA ACCTTCCTTC GTTCTTCGAA
CCTCCGACTT
[SEQ ID No: 13]
CTTATTTAGG TGACACTATA GGGGGAACTA CGACGGTATC TGA
[SEQ ID No: 14]
CTTAATTAAC CCTCACTAAA GGGGGAACTA CGACGG TAT CTG A
[SEQ ID No: 15]
GGCACGTACG AATATACCAC ATACCAAACC TTCCTTCGTA
CGTGCCGCCA AGAATGTTTT CATTAATCAAG

Thus, in a preferred embodiment, the one or more primers are encoded by a nucleotide sequence substantially set out in any one of SEQ ID Nos: 9 to 15, or a fragment or variant thereof.

In another embodiment, the target nucleic acid sequence is from Influenza-A. One embodiment of the Influenza-A target nucleic acid sequence is referred to herein as SEQ ID No: 16, as follows:

[SEQ ID No: 16]
GAGAAUCCAG CACACAAGAG UCAACUGGUG UGGAUGGGAU
GCCAUUCUGC CGCAUUUGAA GAUCUAAGAG UAUUGAGCUU
CAUCAAAGGG ACGAAGGUGG UCC

Thus, in a preferred embodiment, the Influenza-A target nucleic acid sequence comprises or consists of a nucleotide sequence substantially set out as SEQ ID No: 16, or a fragment or variant thereof.

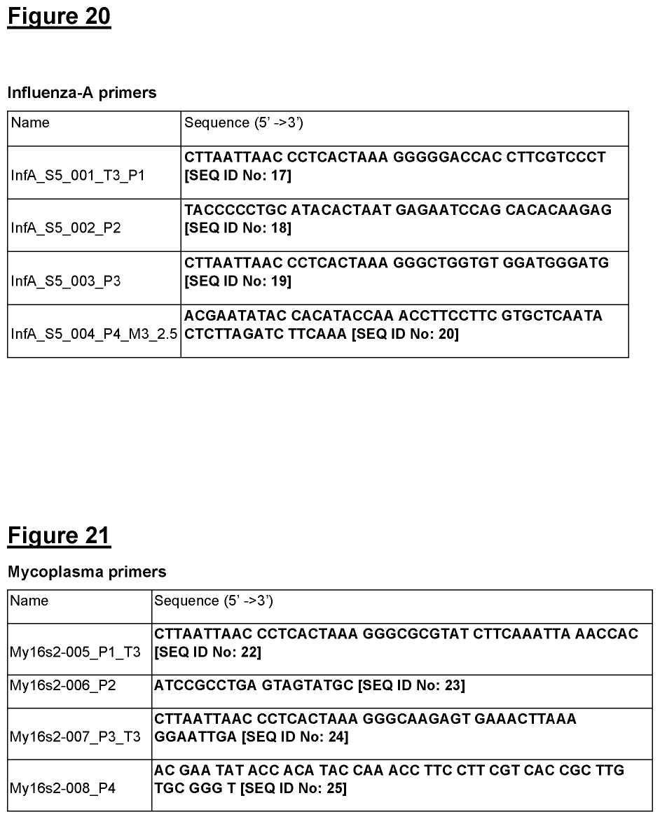

Preferably, in this embodiment, the one or more primers are capable of hybridising to the nucleotide sequence as substantially set out in SEQ ID No: 16, or a fragment or variant thereof. Examples of such primers are listed in FIG. 20.

Accordingly, in one embodiment, the nucleotide sequences encoding the one or more Influenza-A primers are referred to herein as SEQ ID Nos: 17 to 20, as follows:

{SEQ ID No: 17]
CTTAATTAAC CCTCACTAAA GGGGGACCAC CTTCGTCCCT
[SEQ ID No: 18]
TACCCCCTGC ATACACTAAT GAGAATCCAG CACACAAGAG
[SEQ ID No: 19]
CTTAATTAAC CCTCACTAAA GGGCTGGTGT GGATGGGATG
[SEQ ID No: 20]
ACGAATATAC CACATACCAA ACCTTCCTTC GTGCTCAATA
CTCTTAGATC TTCAAA

Thus, in a preferred embodiment, the one or more primers are encoded by a nucleotide sequence substantially set out in any one of SEQ ID Nos: 17 to 20, or a fragment or variant thereof.

In another embodiment, the target nucleic acid sequence is from Mycoplasma. One embodiment of the Mycoplasma target nucleic acid sequence is referred to herein as SEQ ID No: 21, as follows:

[SEQ ID No: 21]
ATCCGCCTGA GTAGTATGCT CGCAAGAGTG AAACTTAAAG
GAATTGACGG GAACCCGCAC AAGCGGTGGA GCATGTGGTT
TAATTTGAAG ATACGCG

Thus, in a preferred embodiment, the Mycoplasma target nucleic acid sequence comprises or consists of a nucleotide sequence substantially set out as SEQ ID No: 21, or a fragment or variant thereof.

Preferably, in this embodiment, the one or more primers are capable of hybridising to the nucleotide sequence as substantially set out in SEQ ID No: 21, or a fragment or variant thereof. Examples of such primers are listed in FIG. 21.

Accordingly, in one embodiment, the nucleotide sequences encoding the one or more Mycoplasma primers are referred to herein as SEQ ID Nos: 22 to 25, as follows:

[SEQ ID No: 22]
CTTAATTAAC CCTCACTAAA GGGCGCGTAT CTTCAAATTA
AACCAC
[SEQ ID No: 23]
ATCCGCCTGA GTAGTATGC
[SEQ ID No: 24]
CTTAATTAAC CCTCACTAAA GGGCAAGAGT GAAACTTAAA
GGAATTGA
[SEQ ID No: 25]
AC GAA TAT ACC ACA TAC CAA ACC TTC CTT CGT CAC
CGC TTG TGC GGG T

Thus, in a preferred embodiment, the one or more primers are encoded by a nucleotide sequence substantially set out in any one of SEQ ID Nos: 22 to 24, or a fragment or variant thereof.

Preferably, the method or kit comprises the use of a plurality of deoxynucleotide triphosphates (dNTPs) and nucleotide triphosphates (NTPs). Preferably, the amplification step comprises the use of a plurality of deoxynucleotide triphosphates (dNTPs) and nucleotide triphosphates (NTPs).

Preferably, the plurality of deoxynucleotide triphosphates (dNTPs) are selected from the group consisting of dATP, dGTP, dCTP and/or dTTP. dNTPs are the building blocks of DNA.

Preferably, the plurality of nucleotide triphosphates (NTPs) are selected from the group consisting of ATP, GTP, CTP and/or UTP. NTPs are the building blocks of RNA.

As discussed in Example 5, the inventors investigated the contribution of Tris-HCl levels to the nested Mango-NASBA assay. Surprisingly, the inventors discovered that an increase in Tris-HCl reduced the amplification levels of Mango RNAs, and 20 mM Tris-HCl appeared to be the optimal amount for the NASBA reaction.

Accordingly, in a preferred embodiment, the amplification comprises contacting the sample with between 1 and 59 mM Tris HCl, between 2 and 55 mM Tris HCl, between 4 and 50 mM Tris HCl, between 6 and 45 mM Tris HCl, between 8 and 40 mM Tris HCl, between 10 and 35 mM Tris HCl, between 12 and 30 mM Tris HCl, between 14 and 25 mM Tris HCl, or between 16 and 20 mM Tris HCl. More preferably, the amplification comprises contacting the sample with between 4 and 55 mM Tris HCl, between 6 and 45 mM Tris HCl, between 8 and 35 mM Tris HCl, between 10 and 30 mM Tris HCl, between 12 and 28 mM Tris HCl, between 14 and 26 mM Tris HCl, between 16 and 24 mM Tris HCl, or between 18 and 22 mM Tris HCl. Most preferably, the amplification comprises contacting the sample with 20 mM Tris-HCl.

Accordingly, in a preferred embodiment, the amplification comprises contacting the sample with between 5 and 59 mM Tris HCl. More preferably, the amplification comprises contacting the sample with between 10 and 59 mM Tris HCl, between 15 and 59 mM Tris HCl, or between 18 and 59 mM Tris HCl. Alternatively, the amplification comprises contacting the sample with between 1 and 55 mM Tris HCl, between 1 and 50 mM Tris HCl, between 1 and 40 mM Tris HCl, between 1 and 30 mM Tris HCl, or between 1 and 25 mM Tris HCl. Most preferably, the amplification comprises contacting the sample with between 1 and 22 mM Tris-HCl.

It will be appreciated that the kit according to the third aspect may comprise any one of the above specified concentrations of Tris HCl.

The inventors also investigated the contribution of pH to the NASBA reaction (see Example 6), and discovered that pH 8.0 indicated the best amplification levels and response speeds.

Accordingly, in a preferred embodiment, the amplification step is conducted at a pH of between 6.8 and 9.0, between 7.0 and 8.8, between 7.2 and 8.6, between 7.4 and 8.4, or between 7.6 and 8.2.

In one embodiment, the amplification step is conducted at a pH of between 6.8 and 8.8, between 6.8 and 8.7, between 6.8 and 8.6, between 6.8 and 8.5, between 6.8 and 8.4, between 6.8 and 8.3, between 6.8 and 8.2, between 6.8 and 8.1, or between 6.8 and 8.0. In another embodiment, the amplification step is conducted at a pH of between 7.0 and 9.0, between 7.1 and 9.0, between 7.2 and 9.0, between 7.3 and 9.0, between 7.4 and 9.0, between 7.5 and 9.0, between 7.6 and 9.0, between 7.7 and 9.0, between 7.8 and 9.0, between 7.9 and 9.0, or between 8.0 and 9.0. Preferably, the amplification step is conducted at a pH of between 7.5 and 8.5, between 7.6 and 8.4, between 7.7 and 8.3, between 7.8 and 8.2, or between 7.9 and 8.1. Most preferably, the amplification step is conducted at a pH of 8.0.

It will be appreciated that the reagents of the kit according the third aspect may comprise any one of the above specified pH levels.

As discussed in Example 7, the inventors also investigated the contribution of spermidine to the NASBA reaction, and discovered that the presence of spermidine enhances the amplification and response time of the original NASBA reaction.

Accordingly, preferably the amplification step comprises contacting the sample with at least 0.1 mM spermidine, at least 0.25 mM spermidine or at least 0.5 mM spermidine. Preferably, the amplification step comprises contacting the sample with at least 0.75 mM spermidine, at least 1 mM spermidine or at least 1.25 mM spermidine. Preferably, the amplification step comprises contacting the sample with at least 01.5 mM spermidine, at least 1.75 mM spermidine or at least 2 mM spermidine.

In a preferred embodiment, the amplification step comprises contacting the sample with between 1 and 20 mM spermidine, between 1.2 and 15 mM spermidine, between 1.4 and 10 mM spermidine, between 1.6 and 5 mM spermidine, between 1.8 and 4 mM spermidine, or between 2 and 3 mM spermidine.

In one embodiment, the amplification comprises contacting the sample with between 1.2 and 20 mM spermidine, between 1.4 and 20 mM spermidine, between 1.6 and 20 mM spermidine, between 1.8 and 20 mM spermidine, between 2 and 20 mM spermidine, between 1 and 15 mM spermidine, 1.2 and 15 mM spermidine, between 1.4 and 15 mM spermidine, between 1.6 and 15 mM spermidine, between 1.8 and 15 mM spermidine, between 2 and 15 mM spermidine, between 1 and 10 mM spermidine, 1.2 and 10 mM spermidine, between 1.4 and 10 mM spermidine, between 1.6 and 10 mM spermidine, between 1.8 and 10 mM spermidine, between 2 and 10 mM spermidine. In one embodiment, the amplification step comprises contacting the sample with between 1.2 and 5 mM spermidine, between 1.4 and 5 mM spermidine, between 1.6 and 5 mM spermidine, between 1.8 and 5 mM spermidine, or between 2 and 5 mM spermidine. Alternatively, the amplification step comprises contacting the sample with between 1 and 4.5 mM spermidine, between 1 and 4 mM spermidine, between 1 and 3.5 mM spermidine, between 1 and 3 mM spermidine, between 1 and 2.5 mM spermidine, or between 1 and 2 mM spermidine. Most preferably, the amplification step comprises contacting the sample with 2 mM spermidine.

It will be appreciated that the kit according to the third aspect may comprise any one of the above specified concentrations of spermidine.

In the original Mango-NASBA protocol, the annealing step was included at 65° C. for 10 minutes before adding the enzyme mix at 41° C. Since the annealing temperature and NASBA reaction temperature are different, the annealing step increases the assay time. To understand the effect of the annealing step in the NASBA reaction, the inventors compared the Mango signals with and without the annealing step and surprisingly discovered that the annealing does not enhance the NASBA reaction. Additionally, the inventors found that approximately 37° C. was the optimum temperature for the amplification of Mango signals.

Accordingly, in a preferred embodiment, the amplification step is conducted at a fixed temperature. Preferably, the amplification step is conducted at a fixed temperature of between 30 and 70° C., between 31 and 60° C., between 32 and 50° C., between 33 and 40° C., between 34 and 39° C., or between 35 and 38° C.

In one embodiment, the amplification step is conducted at a fixed temperature of between 30 and 64° C., between 30 and 58° C., between 30 and 54° C., between 30 and 50° C., between 30 and 46° C., between 30 and 42° C., or between 30 and 38° C. In another embodiment, the amplification step is conducted at a fixed temperature of between 31 and 70° C., between 32 and 70° C., between 33 and 70° C., between 34 and 70° C., between 35 and 70° C., or between 36 and 70° C. Most preferably, the amplification step is conducted at a fixed temperature of 37° C.

The inventors also discovered that the human body could be utilised to incubate the NASBA reaction mixture. Accordingly, in one embodiment, the amplification step comprises incubation by holding the reaction mixture in an individual's hands.

It will be appreciated that the kit according to the third aspect may be used at any one of the above specified temperatures.

The sample is preferably a biological bodily sample taken from the test subject. Detecting the presence of the target nucleic acid sequence in the sample is therefore preferably carried out in vitro. The sample may comprise tissue, blood, plasma, serum, spinal fluid, urine, sweat, saliva, sputum, tears, breast aspirate, prostate fluid, seminal fluid, vaginal fluid, stool, cervical scraping, amniotic fluid, intraocular fluid, mucous, moisture in breath, animal tissue, cell lysates, tumour tissue, hair, skin, buccal scrapings, nails, bone marrow, cartilage, prions, bone powder, ear wax, or combinations thereof. The sample may be a biopsy. Preferably, the sample is blood. Preferably the sample comprises cultured cells. Most preferably, the sample is saliva.

In another embodiment, the sample may be contained within the test subject, which may be an experimental animal (e.g. a mouse or rat) or a human, wherein the method is an in vivo based test. Alternatively, the sample may be an ex vivo sample or an in vitro sample. Therefore, the cells being tested may be in a tissue sample (for ex vivo based tests) or the cells may be grown in culture (an in vitro sample). Preferably, the biological sample is an ex vivo sample.

In some embodiments, the amplification may be multiplexed. Advantageously, this allows the simultaneous detection of multiple target nucleic acids within the same reaction.

Accordingly, in one embodiment, the primer pairs are distinct for each target nucleic acid to be amplified. For example, in one embodiment, the sample may be contacted with two primer pairs, which preferentially amplify two distinct target nucleic acid sequences. Alternatively, in another embodiment, the sample may be contacted with three primer pairs, which preferentially amplify three distinct target nucleic acid sequences. Preferably, in this embodiment, different fluorescent molecules having distinct emission spectra may be used.

It will be appreciated that the kit according to the third aspect may comprise primer pairs as described above, which are distinct for different target nucleic acids, for use in a multiplexed amplification reaction.

In some embodiments, the amplification step further comprises contacting the sample with MgCl₂, KCl, DMSO, ddH₂O and/or DTT. Accordingly, in one embodiment, the kit comprises MgCl₂, KCl, DMSO, ddH₂O and/or DTT. Preferably, the MgCl₂, KCl, DMSO, ddH₂O and/or DTT are pre-mixed with the Tris-HCl and/or spermidine to form the “NASBA buffer”.

In some embodiments, the amplification step further comprises contacting the sample with Superscript III RT, RNase H and/or BSA. Accordingly, in one embodiment, the kit comprises Superscript III RT, RNase H and/or BSA. Preferably, the Superscript III RT, RNase H, and/or BSA are pre-mixed with the polymerase, NASBA buffer, and/or ddH₂O to form the “enzyme mix”.

In some embodiments, the amplification step is performed in a pre-mixed “reaction mixture”. Preferably, the reaction mixture comprises the NASBA buffer, NTPs, dNTPs, one or more primers, the sample comprising the target nucleic acid sequence (e.g. saliva), ddH₂O, TO1-B and the “enzyme mix”.

As discussed in Example 8, the inventors discovered that 10% saliva (v/v) to the total reaction volume is optimal in the Mango-NASBA assay. Accordingly, in one embodiment, the reaction mixture comprises between 1 and 50% saliva (v/v), between 2 and 45% saliva (v/v), between 3 and 40% saliva (v/v), between 4 and 35% saliva (v/v), between 5 and 30% saliva (v/v), between 6 and 25% saliva (v/v), between 7 and 20% saliva (v/v), or between 8 and 15% saliva (v/v). In one embodiment, the reaction mixture comprises between 1 and 45% saliva (v/v), between 1 and 40% saliva (v/v), between 1 and 35% saliva (v/v), between 1 and 30% saliva (v/v), between 1 and 25% saliva (v/v), between 1 and 20% saliva (v/v), between 1 and 15% saliva (v/v), or between 1 and 12% saliva (v/v). In another embodiment, the reaction mixture comprises between 2 and 50% saliva (v/v), between 3 and 50% saliva (v/v), between 4 and 50% saliva (v/v), between 5 and 50% saliva (v/v), between 6 and 50% saliva (v/v), between 7 and 50% saliva (v/v), between 8 and 50% saliva (v/v), or between 9 and 50% saliva (v/v). Most preferably, the reaction mixture comprises 10% saliva (v/v).

It will be well understood by the skilled person that any one of the amplification parameters, such as nucleotide concentration and/or buffer concentration, can also be optimized to improve the Mango-NASBA assay. An exemplary NTP mix, NASBA buffer, enzyme mix and reaction mix are provided in Tables 1 to 4.

In some embodiments, the method further comprises quantifying the target nucleic acid. The quantification step occurs after the amplification step of the method according to the first and second aspect. In one embodiment, the kit comprises means for quantifying the target nucleic acid. The quantification may comprise measuring the intensity of the fluorescent signal, to determine the concentration of the target nucleic acid in the sample.

The kit and/or method may comprise the use of a positive control and/or a negative control against which the fluorescent signal may be compared. For example, a negative control sample is a sample which does not contain the target nucleic acid sequence. A positive control sample is a sample which does contain the target nucleic acid sequence.

Accordingly, in one embodiment, the method further comprises comparing the fluorescent signal with the fluorescent signal of a positive and/or negative control. In one embodiment, an increase in fluorescent signal compared to the negative control suggests that the sample does contain the target nucleic acid sequence. In another embodiment, a fluorescent signal with an intensity equal to or greater than the positive control suggests that sample does contain the target nucleic acid sequence.

Alternatively, a decrease in fluorescent signal compared to the positive control suggests that the sample does not contain the target nucleic acid. In another embodiment, a fluorescent signal with an intensity equal to or less than the negative control suggests that the sample does not contain the target nucleic acid sequence.

Preferably, the kit comprises a positive control, which contains the target nucleic acid sequence. Additionally, the kit may comprise a negative control, which does not contain the target nucleic acid sequence. In a preferred embodiment, the kit comprises a positive control and a negative control. Preferably, the kit comprises means for comparing the fluorescent signal of the sample with the fluorescent signal of the positive and/or negative control.

Advantageously, by detecting the presence of a target nucleic acid sequence, the methods and kit of the invention provide a rapid and sensitive means for diagnosing an infectious disease. Preferably, the methods and/or kit of the invention are useful for enabling a clinician to make decisions with regards to the best course of treatment for a subject who is currently suffering from an infectious disease. Preferably, the methods and/or kit are useful for providing a prognosis of the subject's condition, such that treatment for the infectious disease can be administered to the subject.

Accordingly, the difference in fluorescent signal between the sample and the positive and/or negative control, can be used as a diagnostic and/or prognostic marker, suggestive of the subject suffering from an infectious disease. It will be appreciated that if the sample has an increased fluorescent signal compared to the negative control, or a fluorescent signal with an intensity equal to or greater than the positive control, then the subject suffers from the infectious disease. Alternatively, if the sample has a decreased fluorescent signal compared to the positive control, or a fluorescent signal with an intensity equal to or less than the negative control, then the subject does not suffer from the infectious disease.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID Nos: 1-16 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the 20) alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage of identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage of identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:

FIG. 1 is a schematic of the prior art nested Mango-NASBA assay process. The nested Mango-NASBA assay comprises an outer primer pair, whose products are then diluted and fed into a second inner Mango-NASBA reaction, reducing the production of artefacts. The addition of the Mango aptamer template to primer PD results in the production of an RNA product containing a fluorescent Mango tag.

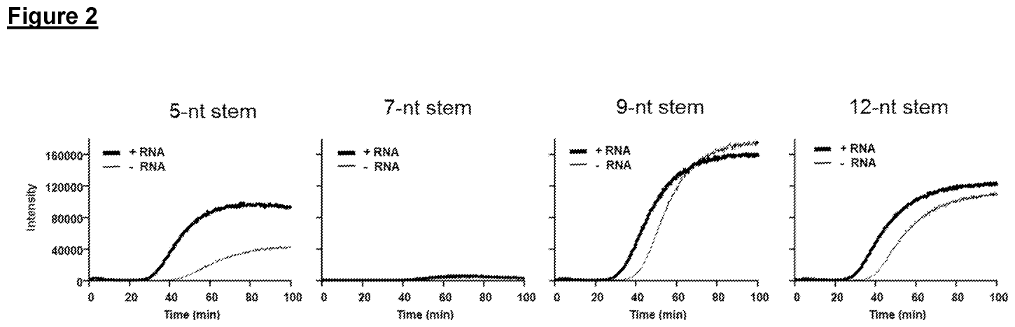

FIG. 2 shows optimization of P4-Mango primer for one embodiment of the method of the invention comprising a NASBA reaction. The NASBA reaction was performed with the conventional incubation steps in the presence of T7 RNA polymerase (180 U per reaction, NEB: M0251L), 25 nM Primers, 50 nM TO1-B, and 40 ng HEK293T RNAs.

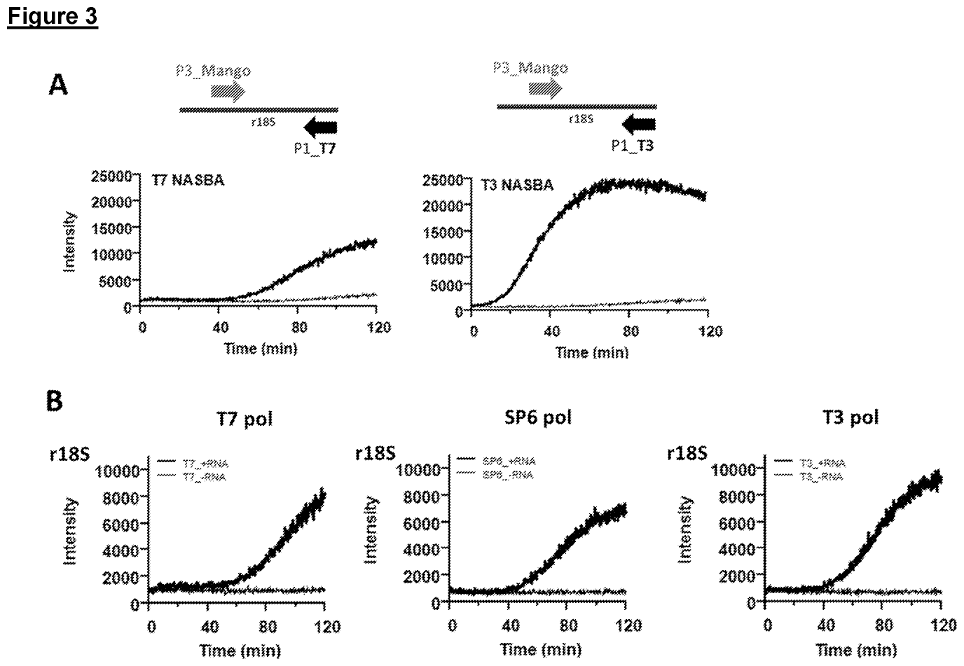

FIG. 3 illustrates a comparison of three different RNA polymerases in the Mango-NASBA reaction (not nested). (A) T7 or T3 RNA polymerase-mediated NASBA reactions for the amplification of 18S ribosomal RNA (18S-rRNA) with the set of primers. (B) The response time and amplification levels with three different RNA polymerases (T7, T3 or SP6) in the Mango-NASBA reaction (not nested). All reaction conditions and results are comparable (180 U of RNA polymerases, 25 nM of primers, 50 nM of TO1-B, 35 ng of HEK293T tot-RNA per reaction with the conventional reactions steps as described above).

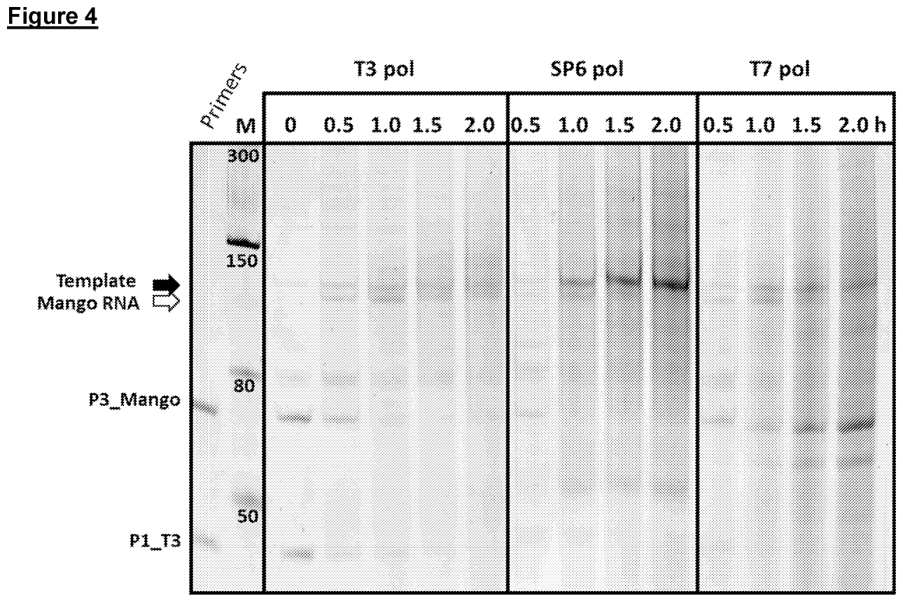

FIG. 4 shows PAGE for the detection of Mango-NASBA RNA transcripts over time (0-2 h) with three different RNA polymerases. Left lanes show the two primers (P1_T3 and P3_M) used for T3 pol mediated Mango-NASBA reaction and the marker-ladder (M) in base pairs (ssRNA ladder N0362S). Mango-NASBA reaction was carried out with 35 ng of HEK293T tot-RNA. Mango-NASBA products were collected at each time point and loaded into 15% polyacrylamide gel (PAGE) under denaturing conditions (8 M Urea), and stained with SYBR Gold (S11494). White arrow indicates the expected NASBA product, while black one is supposed to be synthesized DNA templates.

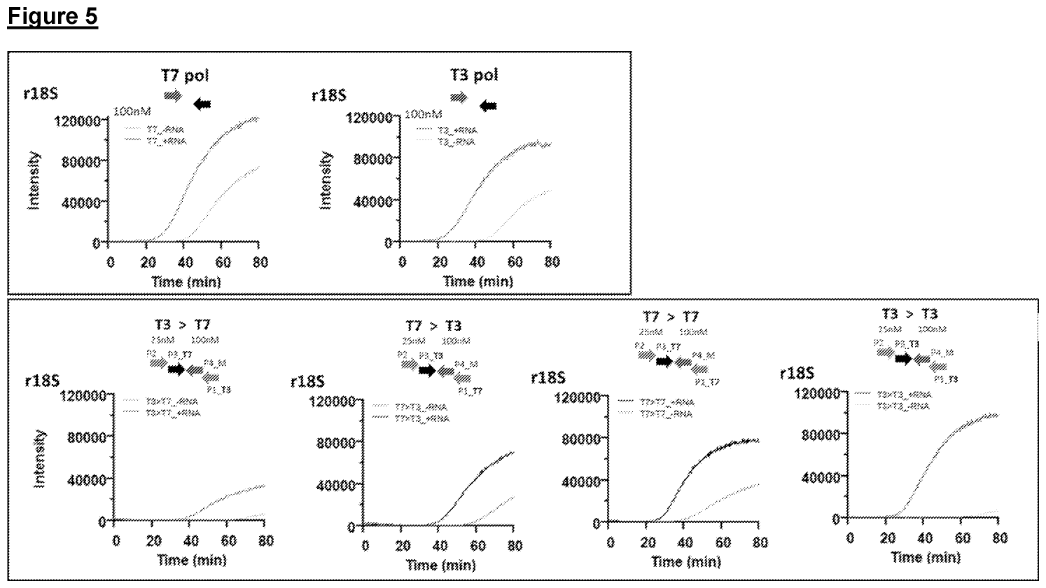

FIG. 5 shows the effect of RNA polymerase combinations on the one-pot nested Mango-NASBA. Top: non-nested Mango-NASBA reaction for 18S-rRNA amplification with either T7 or T3 RNA polymerase. Bottom: One-pot nested NASBA reactions in different combinations using T7 and T3 RNA polymerases. The reactions were carried out with 35 ng of HEK293T tot-RNA and 50 nM TO1-B at the standard Mango-NASBA conditions.

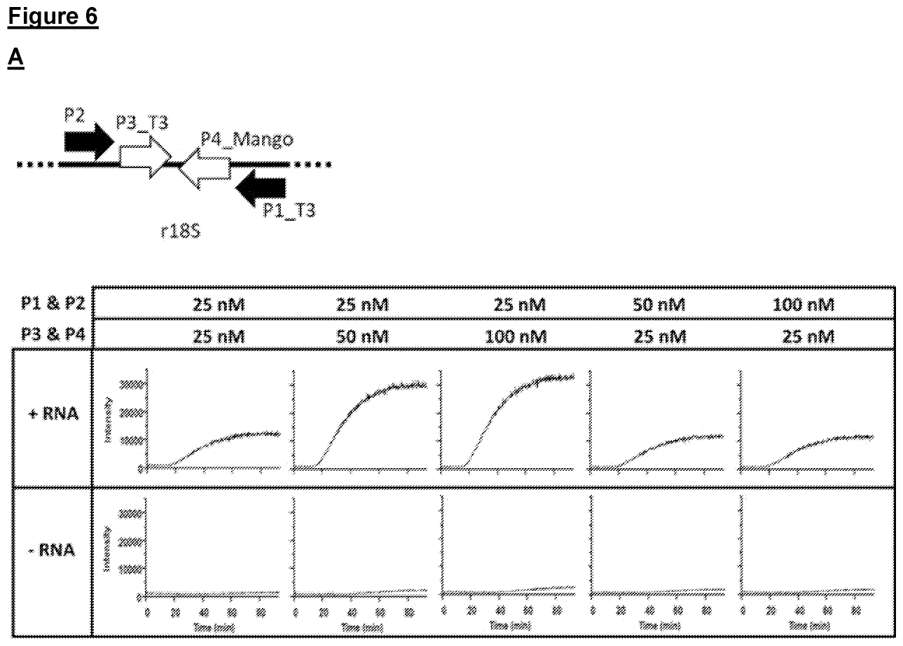

FIG. 6 demonstrates the contribution of primer concentration to the one-pot nested-NASBA reaction. Left arrows show the primer sets used for the amplification of 18S-rRNA. Outer and inner primer concentrations (P1-P4) were systematically titrated between 25-100 nM (FIG. 6A) and between 5-100 nM (FIG. 6B) for the single-pot nested Mango-NASBA reaction. The reaction conditions were 20 mM Tris-HCl (pH 8.0), 100 ng HEK tot-RNA, 180U T3 RNA polymerase, and 2 mM spermidine. The images were taken after 1 h 20 min.

FIG. 7 demonstrates the contribution of Tris-HCl amount in the NASBA reaction. The pH value was fixed at pH 8.8 and the NASBA reaction for r18S amplification was performed with different Tris-HCl levels. The bottom row shows the amplifications in the range of 40-53.3 mM with T3 pol. The top row shows the results in the range of 0-40 mM with T7 pol. According to the results, 20 mM Tris-HCl is optimal for the NASBA reaction.

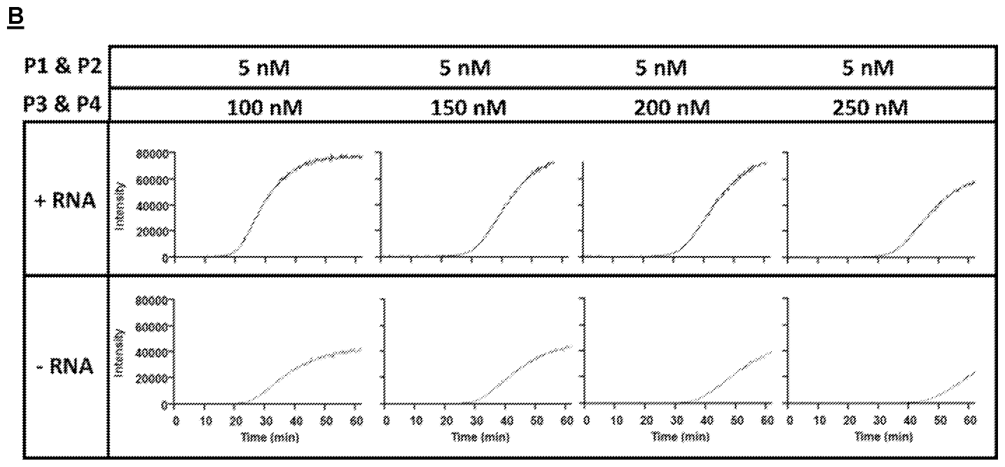

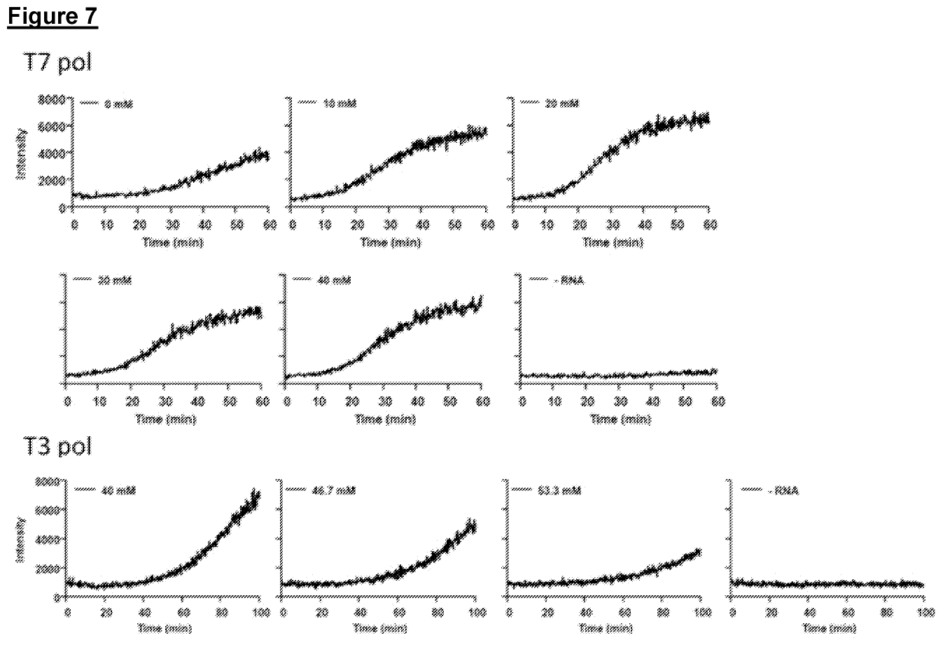

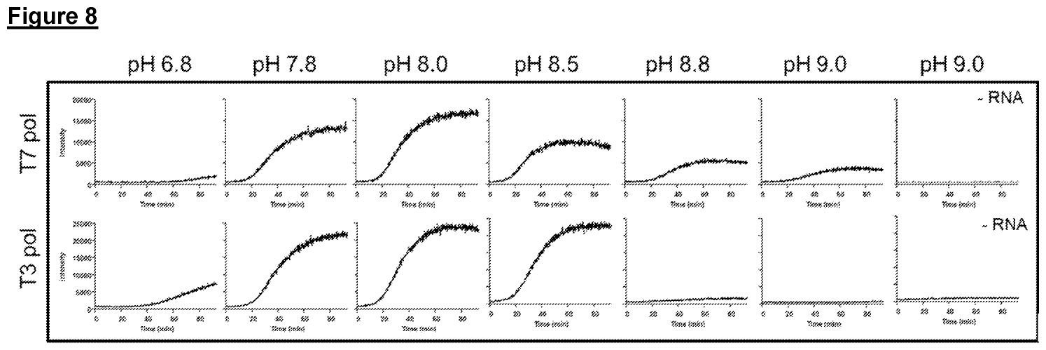

FIG. 8 illustrates the effect of pH on the NASBA reaction. The titration of PH levels with 40 mM Tris-HCl in the presence of T7 or T3 polymerase using r18S primers. In both cases, the NASBAs with pH 8.0 indicate optimal amplification levels and response speeds. The target is 18S-rRNA.

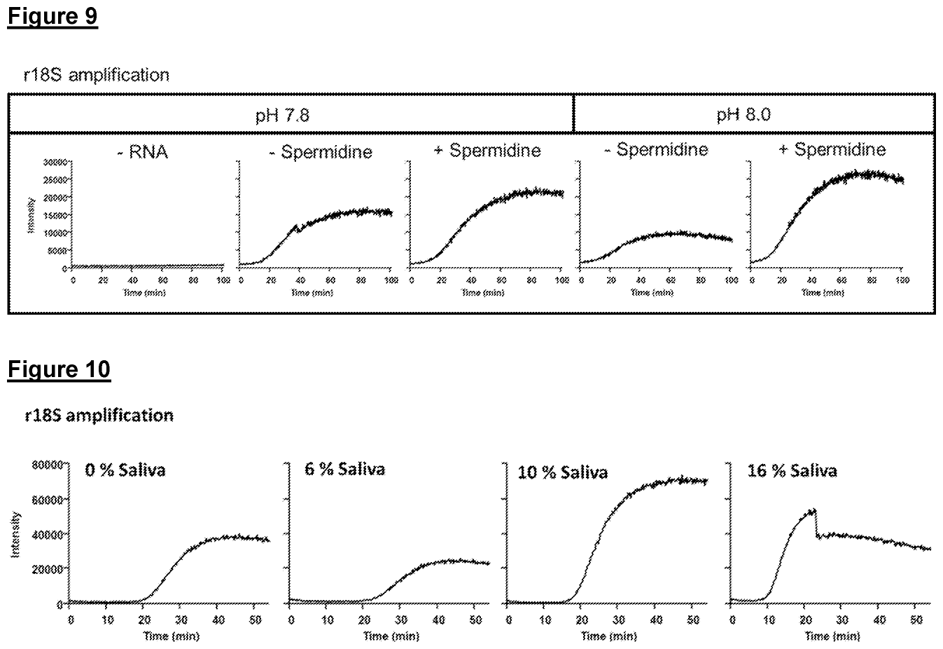

FIG. 9 shows the effect of spermidine concentration on the NASBA reaction. The presence of spermidine enhances the amplification and response time upon the conditional NASBA reaction. 20 mM of Tris-HCl, 25 nM of Primers, 200 ng of HeLa RNAs, and T7 pol were used. The results are reproducible with HEPES based buffers (not shown).

FIG. 10 shows the detection of endogenous RNAs in saliva upon the optimized nested NASBA method. 18S ribosomal RNAs naturally expressed in cells in saliva were successfully detected upon the optimized single-pot nested NASBA condition (20 mM Tris-HCl, pH 8.0, and 2 mM Spermidine).

FIG. 11 shows the detection of viral RNAs with the optimized condition. Left: titration of viral RNA (vRNA) fragments (Fragment 2, F2) in a single-step Mango-NASBA reaction. Primer amounts ([P1] & [P2]=25 nM, [P3] & [P4]=100 nM). Right: 2 ng of viral RNA fragments were used as a positive control in the culture medium. While the cultured SARS-CoV-2 viruses were successfully detected with the treatment of transport buffer, no treatment resulted in no amplification. 4% of cultured viruses (10⁶ TCID50/ml) were applied to the NASBA reaction. With transport buffer treatment, the reaction mixture contains 50 mM of Guanidine-HCl in the final concentration. Both incubation steps of the NASBA reactions were done with the conventional method. Saliva is not included in this experiment.

FIG. 12 demonstrates the contribution of the annealing step and the optimal temperature for the NASBA reaction. Left: arrows show the primer concentrations (P1 & P2/P3 & P4) used for the amplification. The bottom graph show the effect of the annealing step in the single-pot nested NASBA, suggesting that the annealing step does not enhance the single-tube-nested NASBA reaction, and can be omitted. The right images indicate the incubation temperature optimization in the NASBA reaction. The NASBA at around 37° C. produces the better result than 41° C. that has been used for the conventional NASBA reaction.

FIG. 13 illustrates NASBA amplification with incubation via human body. The NASBA reaction mix was incubated by grabbing with a hand. 200 pg of RNA (F2) per reaction. P1 & P2 primers were 25 nM, while the P3 & P4 primers were 100 nM. The incubation time was 30 min.

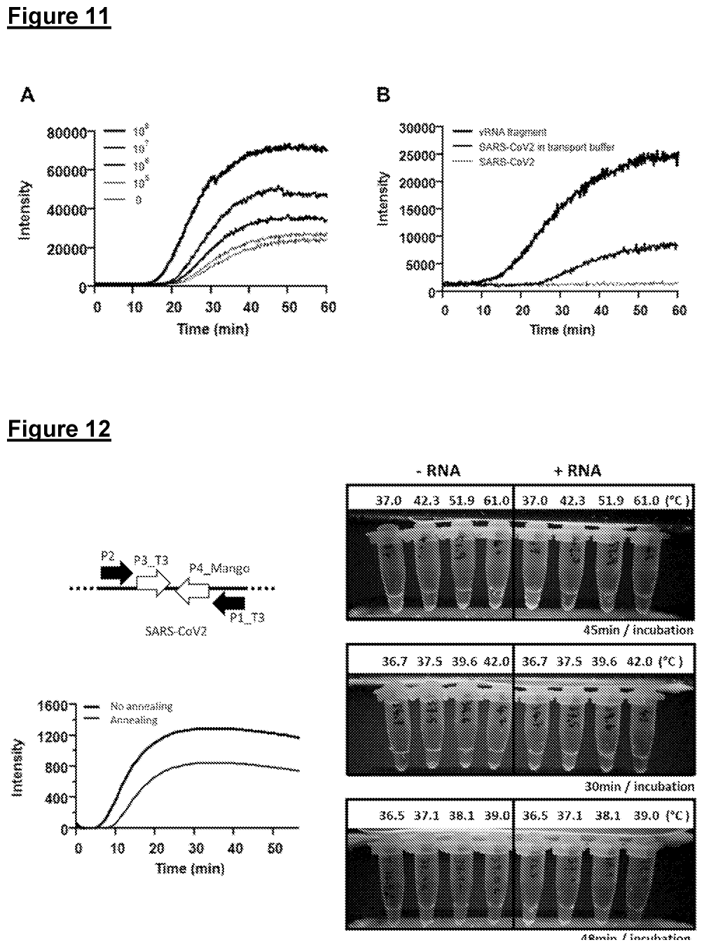

FIG. 14 shows detection of limitation of the one-pot Mango NASBA reaction. The RNA amount was titrated and the detection limitation of this assay was determined. The left shows the results of titration experiments. The coefficients of the gradient were calculated from the linear section of curves in the left panel. The right diagram shows each coefficient value to the corresponding copy number per microliter.

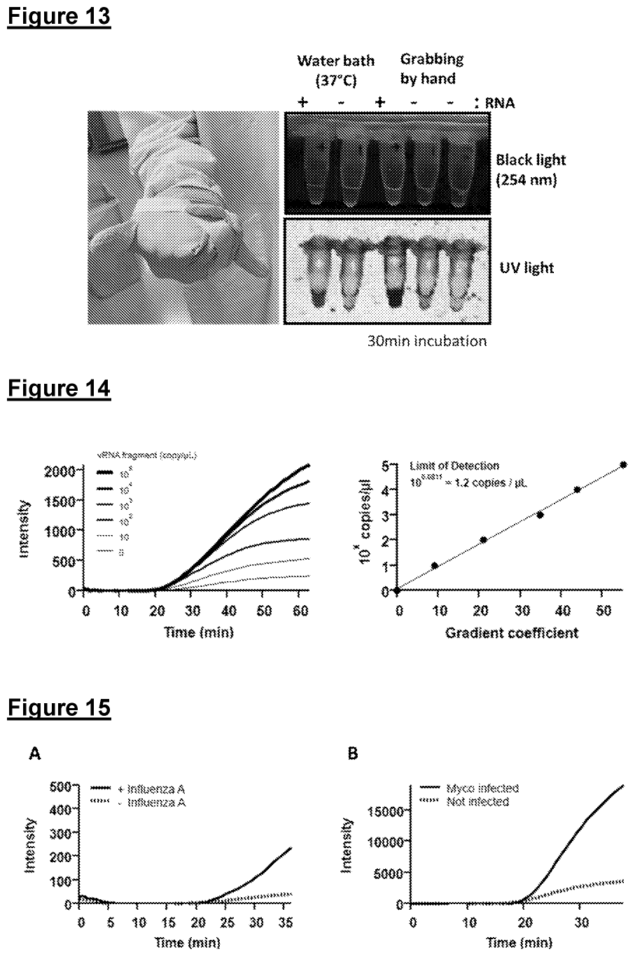

FIG. 15 shows the detection of influenza A virus and Mycoplasma. (A) A RNA segment of Influenza A virus was amplified with Pepper RNA aptamer from the cultured Influenza A viruses. (B) Mycoplasma detection with Mycoplasma infected cells.

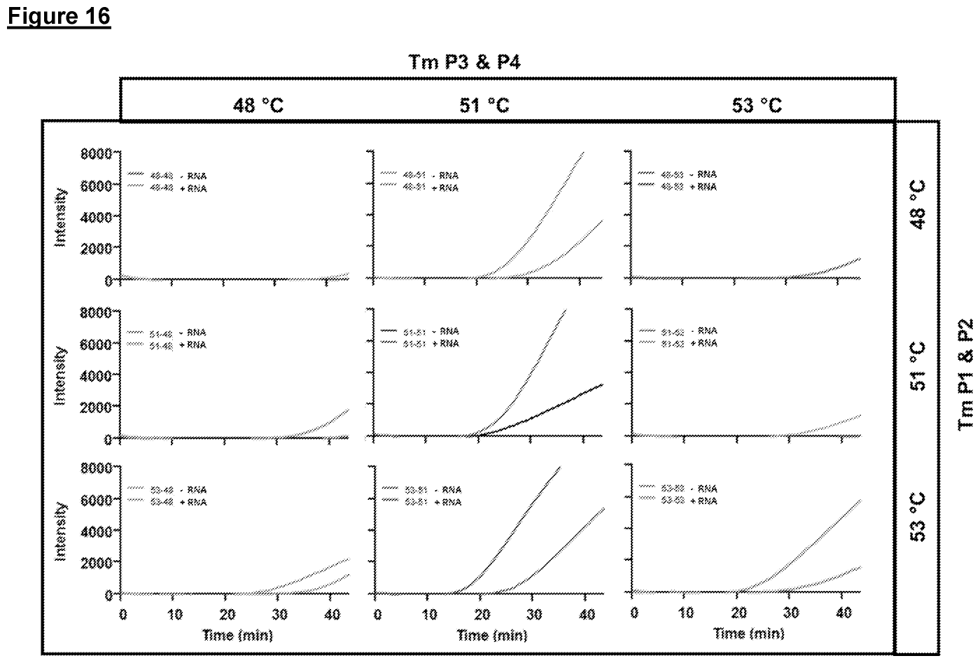

FIG. 16 illustrates the systematic analysis of primer's melting temperature for the NASBA reaction. The melting temperature of P1&2 and P3&P4 primer sets were changed among 48, 51, and 53° C., and the contribution of primer's melting temperature in the NASBA reaction was systematically analyzed. C: Control (minus RNA), RNA: Fragment2 200 pg/reaction.

FIG. 17 illustrates RNA detection in blood samples. (A-C) F2 RNA fragments spiked into a blood sample were amplified by Mango-NASBA method with different buffer conditions. After applying the buffers, the total blood samples were spun for 5 min at 21,000× g, and the supernatants were used for the assays. SARS2-CoV2 template RNAs was 200 pg in reaction. (E-F) Endogenous ribosomal 18S RNA detection by NASBA assay. (E) r18S detection with blood supernatant. (F) Boiled blood samples (95° C./5 min), enhancing the amplification of Mango signals.

FIG. 18 shows primers capable of hybridising to a SARS-CoV-2 target nucleic acid sequence.

FIG. 19 shows primers capable of hybridising to an 18S ribosomal RNA target sequence.

FIG. 20 shows primers capable of hybridising to an influenza A target nucleic acid sequence.

FIG. 21 shows primers capable of hybridising to a mycoplasma target nucleic acid sequence.

### EXAMPLES

### Materials

3× NASBA buffer: 60 mM Tris-HCl (pH 8.0) (Invitrogen AM9855G), 36 mM MgCl₂ (AM9530G), 210 mM KCl (AM9640G), 0.15% DMSO (MP #191418), 6 mM Spermidine (SIGMA S2501-1G), 30 mM DTT (SIGMA D9779-10G).
5× Nucleotide (NT) mix: 50 mM NTPs (NEB N0450L), 25 mM dNTPs (NEB N0447L).
Enzymes: Superscript III Reverse Transcriptase (200 U/μl) (ThermoFisher #1808009), T3 RNA polymerase (50 U/μl) (NEB #M0378S), RNaseH (5 U/μl) (NEB #M297S), BSA (SIGMA A7906-100 G).
Transport buffer: 5M Guanidine-HCl (Life science 0118-500 G), 40% Isopropanol (I9516_500 ml), 0.05% Tween20 (SIGMA P9416_50 ml), 115 mM Sodium Acetate (SIGMA S7988-100 ml).
Lysis buffer: 20 mM Tris-HCl (pH8.0), 2 mM EDTA, 0.5% Triton X-100.

### One-pot Nested NASBA Reaction

TABLE 1
5x NTP-mix

| | | |
|---|---|---|
| Stock | Final | Volume |

| | | | | | | |
|---|---|---|---|---|---|---|
| ATP | 100 | mM | 10 | mM | 40 | μl |
| UTP | 100 | mM | 10 | mM | 40 | μl |
| CTP | 100 | mM | 10 | mM | 40 | μl |
| GTP | 100 | mM | 10 | mM | 40 | μl |
| dNTPs (each) | 10 | mM | 5 | mM | 200 | μl |
| ddH₂O | | | | | 40 | μl |
| Total | | | | | 400 | μl |

TABLE 2
3x NASBA buffer

| | +DMSO (μl) | −DMSO (μl) |
|---|---|---|
| 1M Tris-HCl, pH 8.0 | 30 | 30 |
| 1M MgCl₂ | 18 | 18 |
| 2M KCl | 52.5 | 52.5 |
| DMSO 100% | 75 | |
| ddH₂O | 294.5 | 392 |
| 200 mM Spermidine | 15 | |
| 1M DTT, Fresh | 15 | 7.5 |
| Total (μl) | 500 | 500 |

The 5× NTP-mix (Table 1) and 3× NASBA buffer (Table 2) are prepared first. The inactivation and detection of pathogens in saliva or nasal swab samples can be achieved by the pre-treatment of samples by heating or mixing with the transport buffer. For the heat inactivation, saliva was spat into a 50 mL tube, and 1 mL of saliva was transferred into a 1.5 mL tube. The tubes were incubated with shaking (1,500 rpm) at 95° C. for 2 min by a thermomixer (Eppendorf). After the incubation, the samples were kept on ice until used or stored below −20° C.

TABLE 3
Reaction Mix

| | 1x (μL) | Final (nM) |
|---|---|---|
| 3x NASBA | 10.0 | |
| 5x nucleotide mix | 6.0 | |
| P1 Primer (0.5-2.5 μM) | 0.3 | 5-25 |
| P2 Primer (0.5-2.5 μM) | 0.3 | 5-25 |
| P3 Primer (10 μM) | 0.3 | 100 |
| P4 Primer (10 μM) | 0.3 | 100 |
| RNA template | 1.0 | |
| Saliva | 3.0 | |
| ddH₂O | 0.0 | |
| TO1-B (x100) | 0.3 | |
| Total | 21.5 | |
| Enzyme mix | 8.5 | |
| Total volume | 30.0 | |

TABLE 4
Enzyme Mix

| | | |
|---|---|---|
| Enzymes | Final | 1x (μL) |

| | | | |
|---|---|---|---|
| Superscript III RT (200 U/μL) | 8 | U/reaction | 0.08 |
| T3 RNAP (50 U/μL) | | | 3.6 |
| RNase H (5 U/μL) | 0.05 | U/reaction | 0.02 |
| BSA (20 mg/μL) | 2 | mg/reaction | 0.40 |
| 3x NASBA buffer (no DMSO) | | | 1.40 |
| ddH₂O | | | 3.0 |
| Total | | | 8.50 |

For the preparation of the transport buffer treatment, saliva samples can be transferred into 1.5 mL tubes as described above, but then mixed with the transport buffer at 2:3 ratio (e.g., 400 μL saliva+600 μL transport buffer). The mixtures are further diluted with ddH₂O at 1:5 ratio (e.g., 100 μL of saliva/transport buffer mixture+500 μL of ddH₂O).

The Reaction Mix was prepared as in Table 3 in a 1.5 mL tube, and aliquoted into 200 μL PCR tubes or a 96-well plate for the real-time PCR thermocycler (Bio-Rad: CFX96).

The Enzyme Mix is described as in Table 4, and was prepared in a 1.5 mL tube. After adding 8.5 μL of the Enzyme Mix to each tube or well, the samples are incubated at 37° C. for 30 min. The incubation steps used in the conventional NASBA reaction: The Reaction Mix was pre-heated at 65° C. for 2 min (Annealing), and then pre-incubated at 41° C. for 10 min. After the steps, the Enzyme Mix was applied to the Reaction Mix and the measurement was started.

### Polyacrylamide Gel Electrophoresis (PAGE)

15% acrylamide supplemented with 8M Urea was polymerized in the glass plate chamber. The primers and NASBA products were mixed with Formamide Loading Dye (2×) (1× TBE, 90% Formamide, 1 mM EDTA), and loaded into the wells, after 20 min pre-running. The running power was kept at 24 mW. The gel was stained with SYBR gold (S11494) for 30 min, and canned with an image detector (Amersham Imager 680).

### Methods

1. Prepare 5×NT mix and 3×NASBA buffer as described in Table 1 and Table 2.
2. Pre-treatment of saliva samples for the pathogen inactivation.
   2.1 By heating

Spit the saliva into a sterilized 50-ml falcon tube, and take 1 ml of saliva into a 1.5-ml tube by a P1000 pipette. Place the tube into a thermomixer (Eppendorf), and incubate the tube with 95° C./1500 rpm for 2 minutes. After the incubation, keep the saliva sample on ice until it is used. If it is not used immediately, the heated sample can be stored at −20° C.
2.2 By transport buffer treatment

Spit the saliva into a sterilized 50-ml falcon tube, and take 400 μl of saliva and transfer into 600 μl of the transport buffer in a 1.5-ml tube with a P1000 pipette. Because of the viscosity of saliva, cutting the tip of a 1000-μl tip helps to take an accurate amount of saliva. The final ratio of saliva and transport buffer is three to two respectively. Take 100 μl of the saliva/transport buffer mixture and dilute into 500 μl of water (ddH₂O) in a tube. The relative ratio is one to five, and the final Guanidine-HCl concentration will be 50 mM. Since the volume of transport buffer affects the non-specific signal occurring in the control, the ratio of saliva and transport buffer can be optimized for the reduction of non-specific signals.
3. Preparation of the Reaction Mix

The Reaction Mix can be prepared as in Table 3. The P1 and P2 primer amounts affect the amplification and background signal levels described in FIG. 6, and can be optimized between 5 nM to 25 nM. The P1 and P2 primers create the primary templates, but need to be consumed completely not to interfere with the second NASBA reaction by the P3 and P4 primers. Exceeded P1 and P2 primers in general slowdown the reaction and reduce the amplification levels.

Since T3 RNA polymerase is sensitive to salt amounts, NaOH-adjusted NTPs or dNTPs affect the NASBA reaction. The relative volume of 5×NT mix is 20% of the total reaction volume, and the errors in pH or salt levels of NT mix significantly affects the outcome of the NABSA reaction.

Prepare enough reaction master mixture for the experiment, and aliquot it into 200 μl-PCR tubes or 96-well plate for a real-time PCR thermocycler (Bio-Rad: CFX96).
4. Preparation of the Enzyme Mix

The enzyme master mix is prepared in a 1.5-ml tube as in the Table 4. Add 8.5 μl of the Enzyme Mix to the tubes or wells. The enzyme master mix can be stored at −20° C. Superscript III (ThermoFisher) is better than Superscript IV (ThermoFicher), AMV reverse transcriptase (NEB), and WarmStart reverse transcriptase (NEB) (Data is not shown). The RNaseH activity level affects the NASBA reaction time and amplification levels, but RNaseH appears to not stable in a long-term storage at −20° C.
5. NASBA reaction

The reaction is performed at 37° C., and completed in 30 minutes. Once the reaction mixture is incubated at the reaction temperature, the enzymes lose the activities, and a re-incubation of the mixture no longer amplifies new aptamer RNAs. Incubation longer than 30 minutes generally increases the non-specific signals, causing false positives.

### Results

### Example 1-Optimization of Mango Primers

Relative to the other Mango arrays, Mango III (A10U) array has longer (9-mer) complementary nucleotides at the edges, and forms a double-stranded stem in register. However, longer primers increase the chance of potential unexpected hybridizations, and might enhance non-specific amplifications in the NASBA assay. For this reason, the optimal length of Mango stem nucleotides was systematically determined. The NASBA reaction was performed with the conventional incubation steps, and as illustrated in FIG. 2, the 5-mer stem primer set indicated the best amplification ratio between plus and minus template RNAs.

### Example 2-Comparison of RNA Polymerases

T7, T3, and SP6 RNA polymerases have been commonly used in Molecular Biology, and the functionality of these enzymes was tested in the NASBA assay. All experiments were done in parallel, and the results in FIG. 3 indicate that T3 polymerase amplifies Mango signals more efficiently and rapidly than the other RNA polymerases.

In addition to the detection via a fluorometer, the transcription levels were examined by PAGE detection, as shown in FIG. 4. Surprisingly, the transcription level via T3 was lower than that of the other polymerases, and the result seemingly suggests that the general transcription level and the Mango RNA transcription level are not exactly correlated, and T3 RNA polymerase appears to transcribe Mango RNAs more properly from the synthesized template. Ty and SP6 on the other hand, appear to produce the template DNA rapidly, but the high amounts of DNA templates do not contribute to the production of Mango RNAs. Upon the gel detection, 25 nM of primers were rapidly consumed in the reaction, and this result implies a possibility that an additional set of primers can be mixed for a subsequent nested NASBA reaction in the same tube.

### Example 3-One-pot Nested NASBA Reaction

To test the possibility of a one-pot nested NASBA reaction, the different combinations of T7 and T3 RNA polymerases and the primer sets were mixed, and the Mango signals were monitored over time, as shown in FIG. 5. To this end, mixing two different RNA polymerases neither enhanced the NASBA reactions nor reduced the background signals occurring in the control reactions. The nested Mango reaction only worked when the same RNA promoter sequence were used in the outer (P1) and inner primer (P3), and the same RNA polymerase was used, too. In those cases, the nested NASBA in general reduces the non-specific amplifications.

### Example 4-Optimization of Primer Amounts

The inventors realised that the established single-tube nested NASBA amplifies the target RNAs. Next, the optimal primer concentrations were identified by systematic titration of primers. As expected, the Mango signals were efficiently amplified when the outer primer amount was lower than that of inner primers, as illustrated in FIG. 6A. On the other hand, excess levels of primers (>100 nM) resulted in the reduction of Mango signal amplifications (FIG. 6B).

Since the nested NASBA assay was successful in one tube, the inventors applied this method with intact saliva samples. In COVID-19 tests, the RNA isolation from patient samples is always a time limiting step, and skipping this step will allow for a rapid diagnosis. Saliva entirely eliminates the amplification of Mango signals, and the inventors came to realize that the viscosity and neutral pH (6.2-7.2) of saliva might affect the NASBA reaction if directly applying intact saliva (Data is not shown). The inventors still hoped that the utilization of intact saliva in the NASBA reaction would be possible, and therefore, they attempted to find an optimal NASBA condition by investigating the contribution of other NASBA reaction components.

### Example 5-Optimization of Tris-HCl Amount

Upon the conventional NASBA buffer condition, applying the intact saliva resulted in the reduction of pH. The inventors expected that increasing the amount of Tris-HCl would help maintain the pH value. However, the increase of Tris-HCl reduced the amplification levels of Mango RNAs. To investigate the contribution of Tris-HCl levels, the amount was titrated, and 20 mM Tris-HCl appeared to be optimal for the NASBA reaction, as illustrated in FIG. 7.

### Example 6-pH Contribution

Since adding saliva dropped the pH of NASBA buffer and eliminated the NASBA reaction, the inventors assessed the contribution of the pH to the NASBA reaction. Tris-HCl was kept at 40 mM, and the pH levels were changed (pH 6.8-9.0). The inventors tested both T7 and T3, and in both cases, pH 8.0 indicated the best amplification levels and response speeds (see FIG. 8).

### Example 7-Spermidine Contribution

In general, amine groups of spermidine neutralize and stabilize the phosphate backbones of nucleotides, and spermidine is able to enhance the transcription activity of T7 and T3 RNA polymerases. Therefore, the inventors tested spermidine in the NASBA reaction. 2 mM of spermidine was used in the conventional transcription. As illustrated in FIG. 9, the presence of spermidine enhances the amplification and response time upon the conditional NASBA reaction. The results were reproducible even with Hepes based buffers (Data is not shown).

### Example 8-Trials of Intact Saliva

The optimized nested NASBA condition (20 mM Tris-HCl, pH8.0, and 2 mM Spermidine) was tested for the detection of endogenous 18S ribosomal RNAs occurring in intact saliva. To this end, the endogenous r18S RNAs were successfully detected, and the titration experiment indicated that around 10% saliva to the total reaction volume is optimal in the NASBA assay condition, as shown in FIG. 10.

### Example 9-Detection of SARS-CoV2 RNAs

The optimized condition was applied for the detection of SARS-CoV2 RNAs. 2 ng of viral RNA fragments was used as a positive control in the culture medium. While the cultured SARS-CoV-2 viruses were detected with the treatment of transport buffer, no treatment resulted in no amplification (see FIG. 11). 4% of cultured viruses (1× e6 TCID50/ml) was applied to the NASBA reaction. With transport buffer treatment, the reaction mixture contains 50 mM of Guanidine-HCl in the final concentration. Both incubation steps of the NASBA reactions were done with the conventional condition. Saliva is not included in this experiment.

### Example 10-Optimization of Incubation Steps

In the original protocol (Abdolahzader et al., 2019), the annealing step was included at 65° C. for 10 minutes before applying the Enzyme Mix at 41° C. Since the annealing temperature and NASBA reaction temperature were different, the annealing step in general consumes the assay time. To understand the effect of the annealing step in the NASBA reaction, the inventors compared the Mango signals with and without the annealing step. As illustrated in FIG. 12, it was identified that the annealing step does not enhance the single-tube-nested NASBA reaction, and the Mango NASBA reaction can be done at a single fixed temperature. The inventors further tried to determine the optimal temperature for the reaction, and approximately 37° C. instead of 41° C. showed higher Mango signals.

### Example 11-Potential Incubation Method for the NASBA Assay

The optimal temperature of 37° C. gave the inventors an idea that the human body can be utilized for the incubation of NASBA reaction mixture. To test this idea, a vial of the reaction mixture was grabbed by hand, and wrapped with gloves for 30 min. This experiment was successful and the Mango signal was detected, as shown in FIG. 13.

### Example 12-Limit of Detection

Since all buffer conditions, the incubation method, and the temperature were optimized, the detection limitation of this assay was determined by titrating the amount of target RNAs. The theoretical detection limitation was determined by the coefficients of curves calculated from the linear section of titration curves in the left panel of FIG. 14. When a linear trend line was adjusted to each coefficient value, the theoretical LOD indicated one copy per micro litter. The LOD of RT-LAMP was reported to be 5-100 copies/μL with purified RNAs, and therefore, the LOD of Mango NASBA turned out to be better than the competitive isothermal method.

### Example 13-Detection of Other Pathogens

To demonstrate that the established NASBA method is able to detect other pathogens, the target RNA segment of Influenza A virus was amplified from the cultured Influenza viruses with the NASBA method (see FIG. 15A). The experiment has been done with Pepper RNA aptamer. In the ideal case, different pathogens are detected with different aptamers. In addition to Influenza viruses, mycoplasma in mammalian cells was also successfully detected (FIG. 15B.

### Example 14-Insight Into Primers' Annealing Temperature

Optimising melting temperature (Tm) of primers might help an efficient amplification of Mango signals. For this aspect, the effect of Tm was systematically analysed with different Tm (48, 51, and 53° C.). Ideally, the outer NASBA reaction is done rapidly, and does not disturb the inner NASBA reaction. To this end, the signal difference in the presence and absence of template RNAs was maximized when all primer Tm were 51° C.

### Example 15-RNA Detection in Blood Samples

The possibility of RNA detection in blood samples was investigated. Blood samples were diluted with different buffers, and SARS-CoV2 template RNAs were spiked into the samples. While the total blood materials eliminated the amplification of NASBA reaction (Data is not shown), the supernatants of blood materials, derived after the centrifugation at 21,000×g for 5 minutes works in the NASBA assay, and amplified the Mango signals. A lysis buffer was found to help the amplification in the reaction. Boiling the blood samples further enhances the NASBA reaction and endogenous 18S ribosomal RNAs were successfully detected (see FIG. 17). Since blood samples are commonly used for different types of clinical diagnosis, the results suggest the potential use of Mango-NASBA in clinical diagnoses.

### Discussion

Compared with other assays, the Mango NASBA assay of the claimed invention is a rapid isothermal reaction performed at 37° C., and the amplified Mango signals can be detectable in less than 15 minutes, with the reaction completed within 30 minutes. Due to the optimal incubation temperature of the assay, the human body can potentially be used as an incubating device, suggesting no need of a special heating device. While the other isothermal methods need a specialized DNA polymerase (Bst DNA polymerase), three enzymes in the NASBA reaction (RNA polymerase, reverse transcriptase, and RNaseH) are not unique, and commonly used in the research of Molecular Biology. These features suggest that the Mango NASBA method enables us to reduce the cost per reaction, and overcome the limitation of resources.

In addition, this NASBA reaction is compatible with intact saliva, and can detect the target RNAs in saliva without any pre-treatment. Not only heating but also transport buffer treatment of saliva samples can be compatible with this assay, and the LOD with saliva reveals about 1 copies/μL and can be better than the LOD of RT-LAMP. Since RT-LAMP has already been proved as a point of care COVID-19 diagnosis, the established NASBA is expected to play a role in a point of care COVID-19 diagnosis, as well as in other pathogen diagnosis too. Accordingly, this assay can be easily adapted to detect multiple pathogens simultaneously, and can be utilized at a point of care diagnosis.

### Conclusions

The inventors have established a rapid one-pot pathogen detection method by optimising the Mango RNA aptamer and NASBA reaction. Although several former studies have already attempted to amplify the target RNAs by NASBA, the amplification reactions were extremely slow and suffered from high background signals occurring in the controls. Accordingly, the use of the NASBA method has been limited, especially in the field of pathogen detections. However, the inventors have evaluated the rational of NASBA reaction steps, and established a simple, rapid, isothermal, one-tube method for pathogen detections. This novel NASBA approach therefore provides a new avenue for nucleotide based rapid pathogen detections.

1. A method of detecting a target nucleic acid sequence in a sample obtained from a subject, the method comprising:
amplifying the target nucleic acid sequence by nucleic acid sequence based amplification (NASBA), wherein the amplification comprises contacting the sample with:
(a) one or more primers, wherein at least one of the primers comprises a fluorescent molecule;
(b) one or more polymerases; and
(c) a plurality of deoxynucleotide triphosphates (dNTPs) and/or nucleotide triphosphates (NTPs), and
detecting the target nucleic acid sequence, wherein a fluorescent signal indicates the presence of the target nucleic acid sequence in the sample,
wherein the amplification step is conducted at a fixed temperature of between 30 and 40° C., and optionally wherein the amplification step comprises at least one of the following conditions:
(i) contacting the sample with between 1 and 59 mM Tris HCl;
(ii) contacting the sample with spermidine;
a pH of between 6.8 and 9.0;
(iii) and/or
(iv) the polymerase is T3 RNA polymerase;
wherein the method is conducted in a single step and/or a single pot or container; and
wherein the method does not comprise extracting or purifying the nucleic acid from the sample.

2. A method of diagnosing or prognosing an infectious disease in a subject, the method comprising detecting a target nucleic acid sequence in a sample obtained from a subject, comprising:
amplifying the target nucleic acid sequence by nucleic acid sequence based amplification (NASBA), wherein the amplification comprises contacting the sample with:
(a) one or more primers, wherein at least one of the primers comprises a fluorescent molecule;
(b) one or more polymerases; and
(c) a plurality of deoxynucleotide triphosphates (dNTPs) and/or nucleotide triphosphates (NTPs), and
detecting the target nucleic acid sequence, wherein a fluorescent signal indicates that the subject suffers from an infectious disease or has a negative prognosis,
wherein the amplification step is conducted at a fixed temperature of between 30 and 40° C., and optionally wherein the amplification step comprises at least one of the following conditions:
(i) contacting the sample with between 1 and 59 mM Tris HCl;
(ii) contacting the sample with spermidine;
a pH of between 6.8 and 9.0
(iii) and/or
(iv) the polymerase is T3 RNA polymerase;
wherein the method is conducted in a single step and/or a single pot or container; and
wherein the method does not comprise extracting or purifying the nucleic acid from the sample.

3. A kit for detecting a target nucleic acid sequence in a sample obtained from a subject, the kit comprising:
reagents for amplifying the target nucleic acid sequence by nucleic acid sequence based amplification (NASBA), wherein the reagents comprise:
(a) one or more primers, wherein at least one of the primers comprises a fluorescent molecule;
(b) one or more polymerases; and
(c) a plurality of deoxynucleotide triphosphates (dNTPs) and/or nucleotide triphosphates (NTPs), and
means for detecting the target nucleic acid sequence, wherein a fluorescent signal indicates the presence of the target nucleic acid sequence in the sample,
wherein the kit is used at a fixed temperature of between 30 and 40° C., and optionally wherein the kit comprises at least one of the following:
(i) between 1 and 59 mM Tris HCl;
(ii) spermidine;
a pH of between 6.8 and 9.0;
(iii) and/or
(iv) the polymerase is T3 RNA polymerase;
wherein the kit is configured to detect the target nucleic acid sequence in a single step and/or a single pot or container; and wherein the kit does not comprise means for extracting or purifying the nucleic acid from the sample.

4. The method according to claims 1, wherein the method comprises:
a) at least conditions: (i) and (ii); (i) and (iii); (i) and (iv); (i) and (v); (ii) and (iii); (ii) and (iv); (ii) and (v); (iii) and (iv); (iii) and (v); or (iv) and (v);
b) at least conditions: (i), (ii) and (iii); (i), (ii) and (iv); (i), (ii) and (v); (i), (iii) and (iv); (i), (iii) and (v); (i), (iv) and (v); (ii), (iii) and (iv); (ii), (iv) and (v); (ii), (iv) and (v); or (iii), (iv) and (v);
c) at least conditions: (i), (ii), (iii) and (iv); (i), (ii), (iii) and (v); (i), (ii), (iv) and (v); (i), (iii), (iv) and (v); or (ii), (iii), (iv) and (v); or
d) all of conditions (i), (ii), (iii), (iv) and (v).

5-9. (canceled)

10. The method according to claim 1, wherein the one or more primers comprises a first and second primer, forming a first primer pair.

11. The method according to claim 1, wherein;
a) the one or more primers comprises a first, second, third and fourth primer, wherein the first and second primer form a first primer pair, and the second and third primer form a second primer pair;
b) the fluorescent molecule is a molecular beacon probe or a fluoregenic aptamer, optionally wherein the fluorogenic aptamer is selected from a group of aptamers consisting of:
Mango, Pepper, Peach, Broccoli, Corn, Spinach, Spinach2, Carrot, Radish, RT aptamer, hemin-binding G-quadruplex DNA and RNA aptamers, and a malachite green binding aptamer, optionally wherein the fluorogenic aptamer is a Mango RNA aptamer, optionally wherein the Mango RNA aptamer is a Mango II, Mango III or Mango IV RNA aptamer, optionally wherein the method comprises contacting the sample with thiazole orange-based ligand (T01-Biotin);
c) the primer comprising the fluorescent molecule comprises a terminal stem structure of between 2 bp and 8 bp, or between 3 bp and 7 bp, or between 4 bp and 6 bp, preferably, wherein the primer comprising the fluorescent molecule comprises a terminal stem structure of 5 bp; and/or
d) the concentration of the first and second primer (the first primer pair) is lower than the concentration of the third and fourth primer (the second primer pair), optionally wherein the concentration of the first and second primer is between 1 and 50 nM, between 5 and 45 nM, between 10 and 40 nM, between 15 and 35 nM, or between 20 and 30 nM, and/or wherein the concentration of the third and fourth primer is between 10 and 150 nM, between 20 and 140 nM, between 30 and 130 nM, between 40 and 120 nM, between 50 and 110 nM, between 60 and 100 nM, or between 70 and 90 nM.

12-16. (canceled)

17. The method according to claim 1, wherein:
a) the melting temperature of the one or more primers is between 48 and 54° C., between 49 and 53° C., or between 50 and 52° C., preferably wherein the melting temperature of the one or more primers is 51° C.;
b) the target nucleic acid sequence is a DNA or RNA sequence, optionally wherein the target nucleic acid sequence is selected from chromosomal DNA, mitochondrial DNA, messenger RNA, transfer RNA, ribosomal RNA, small nuclear RNA, micro RNA, small-interfering RNA, viral RNA or extrachromosomal DNA; and/or the target nucleic sequence is from a virus, a bacteria, a mycoplasma, a fungus, an animal, a plant, an alga, a parasite, or a protozoan, preferably wherein the target nucleic acid sequence is from a virus or bacterium;
c) the plurality of deoxynucleotide triphosphates (dNTPs) are selected from the group consisting of dATP, dGTP, dCTP and/or dTTP, and/or where in the plurality of nucleotide triphosphates (NTPs) are selected from the group consisting of ATP, GTP, CTP and/or UTP; and/or
d) the sample comprises tissue, blood, plasma, serum, spinal fluid, urine, sweat, saliva, sputum, tears, breast aspirate, prostate fluid, seminal fluid, vaginal fluid, stool, cervical scraping. amniotic fluid, intraocular fluid, mucous, moisture in breath, animal tissue, cell lysates, tumour tissue, hair, skin, buccal scrapings, nails, bone marrow, cartilage, prions, bone powder, ear wax, or combinations thereof, optionally wherein the sample is a saliva sample, optionally wherein the reaction mixture comprises 10% saliva (v/v)

18-20. (canceled)

21. The method according to claim 1, wherein:
a) the amplification step comprises contacting the sample with between 1 and 59 mM Tris HCl, between 2 and 55 mM Tris HCl, between 4 and 50 mM Tris HCl, between 6 and 45 mM Tris HCl, between 8 and 40 mM Tris HCl, between 10 and 35 mM Tris HCl, between 12 and 30 mM Tris HCl, between 14 and 25 mM Tris HCl, or between 16 and 20 mM Tris HCl;
b) the amplification step comprises a pH of between 6.8 and 9.0, between 7.0 and 8.8, between 7.2 and 8.6, between 7.4 and 8.4, or between 7.6 and 8.2;
c) the amplification step comprises contacting the sample with between 1 and 20 mM spermidine, between 1.2 and 15 mM spermidine, between 1.4 and 10 mM spermidine, between 1.6 and 5 mM spermidine, between 1.8 and 4 mM spermidine, or between 2 and 3 mM spermidine; and/or
d) the amplification step is conducted at a fixed temperature of between 34 and 39° C., or between 35 and 38° C.

22-26. (canceled)

27. The method according to claim 1, wherein the method further comprises quantifying the target nucleic acid comprises means for quantifying the target nucleic acid, optionally wherein the quantification comprises measuring the intensity of the fluorescent signal, optionally wherein the method further comprises comparing the fluorescent signal with the fluorescent signal of a positive and/or negative control.

28. (canceled)
